(19) United States
(12) Patent Application Publication
(10) Pub. No.: US 2008/0184391 A1
(43) Pub. Date: Jul. 31, 2008

(54) Pathogen resistant transgenic plants, associated nucleic acids and techniques involving the same

(76) Inventors: Kuppuswamy Subramaniam, Kanpur (IN); Bindhya Chal Yadav, Kanpur (IN)

Correspondence Address: JYOTI C. IYER, ESQ., 9113 TALLFIELD CT., LANHAM, MD, 20706, US

(21) Appl. No.: 11/783,916

(22) Filed: Apr. 13, 2007

(30) Foreign Application Priority Data
Jan. 29, 2007 (IN) 171/DEL/2007

(51) Int. Cl. A01H 1/00; A01H 5/00; C07H 21/02; C12N 1/20; C12N 15/00

(52) U.S. Cl. 800/279; 435/252.8; 435/320.1; 536/23.1; 800/301

(57) ABSTRACT

A transgenic plant having a nucleic acid molecule of a pathogen, wherein the transgenic plant has increased resistance to infection by the pathogen. The nucleic acid molecule of the pathogen is one of (a) a portion of a polynucleotide coding for a protein of the pathogen; (b) a complementary sequence to (a); (c) a combination of (a) and (b); and (d) a combination of (a) and (b) further having a spacer nucleotide sequence. The protein of the pathogen is one of a protein involved in chromatin remodeling in the pathogen, a protein in a recombination pathway of the pathogen, a protein in a nucleotide repair pathway of the pathogen, a protein for post transcriptional processing of RNA in the pathogen, or combinations thereof.

### RELATED APPLICATIONS

This Application claims priority from Provisional Indian Patent Application Serial No. 171/DEL/2007, filed on Jan. 29, 2007.

### FIELD OF INVENTION

The present disclosure provides compositions and method for modulating the expression of a target gene in a target pathogen through a host plant for the pathogen. In particular, the present disclosure relates to a method of producing transgenic plants having a nucleic acid molecule of the pathogen, whereby transcription of the nucleic acid molecule of the pathogen can inhibit expression of a target gene of the pathogen via an RNA interference mechanism. In some embodiments the target gene for RNAi in the pathogen is an integrase protein of a nematode. In some embodiments the target gene for RNAi in the pathogen is a splicing factor gene of a nematode.

### BACKGROUND OF INVENTION

Several species of nematodes parasitize a wide variety of plants and animals, including humans. Human parasites such as the gut worm Ascaris lumbricoides, hook worms Ancylostoma duodenale and Necator americanus, and the causative agents of the lymphatic filariasis Wuchereria bancrofti and Brugia malayi infect and severely affect the health of about half the world's population (Horton J., Global anthelmintic chemotherapy programs: learning from history, Trends Parasitol 2003; 19:405-409). Similarly, gastrointestinal nematodes (Ostertagia ostertagi) and liver fluke (Fasciola hepatica) infect livestock leading to considerable yield loss in animal husbandry (Loyacano A F, Williams J C, Gurie J, DeRosa A A, Effect of gastrointestinal nematode and liver fluke infections on weight gain and reproductive performance of beef heifers, Vet Parasitol 2002; 107:227-234).

Plant parasites such as the root-knot (Meloidogyne spp.) and cyst (Heterodera and Globodera spp.) nematodes cause significant damage to important crop plants such as legumes, vegetables and cereals in most parts of the world. Annual crop loss due to plant-parasitic nematodes is estimated to be over $125 billion (Chitwood D J., Research on plant-parasitic nematode biology conducted by the United States Department of Agriculture-Agricultural Research Service, Pest Manag Sci 2003; 59:748-753). Despite this enormous impact on world-wide agriculture, currently there is no effective and environmentally safe method available to prevent or treat plant nematode infections (Chitwood D J., Research on plant-parasitic nematode biology conducted by the United States Department of Agriculture-Agricultural Research Service, Pest Manag Sci 2003; 59:748-753). One of the main reasons for this is our limited understanding of the functions of nematode's genes. Since these organisms require a suitable host for their growth and proliferation, it is nearly impossible to culture them in the laboratory. Therefore, it has not been possible to use traditional genetic tools, which are widely used to determine gene function in free-living model organisms, for the functional characterization of parasitic nematode genes.

In many organisms, including the free-living nematode Caenorhabditis elegans, introduction of double stranded RNA (dsRNA) has been observed to deplete the endogenous mRNA that shares a high degree of sequence identity with the introduced dsRNA (Hannon G J., RNAi: A guide to gene silencing, Cold Spring Harbor: Cold Spring Harbor Laboratory Press, 2003). In C. elegans, dsRNA can be introduced by microinjection, soaking or feeding bacteria that express the dsRNA (Fire A, Xu S, Montgomery M K, Kostas S A, Driver S E, Mello C C, Potent and specific genetic interference by double-stranded RNA in Caenorhabditis elegans, Nature 1998; 391:806-811; Timmons L, Court D L, Fire A, Ingestion of bacterially expressed dsRNAs can produce specific and potent genetic interference in Caenorhabditis elegans, Gene 2001; 263:103-112; Subramaniam K, Seydoux G, nos-1 and nos-2, two genes related to Drosophila nanos, regulate primordial germ cell development and survival in Caenorhabditis elegans, Development 1999; 126:4861-4871). However, these approaches could not be easily used in the case of parasitic nematodes. Successful recovery of microinjected juveniles is technically difficult and the juveniles of parasitic nematodes do not take up much material orally from solutions. Urwin et al. overcame this problem by treating juveniles with octopamine which induced oral uptake (Urwin P E, Lilley C J, Atkinson H J, Ingestion of double-stranded RNA by preparasitic juvenile cyst nematodes leads to RNA interference, Mol Plant Microbe Interact 2002; 15:747-752). Since then a few groups have successfully employed this approach to elicit RNAi response in plant-parasitic nematodes (Bakhetia M, Charlton W, Atkinson H J, McPherson M J, RNA interference of dual oxidase in the plant nematode Meloidogyne incognita, Mol Plant Microbe Interact 2005; 18:1099-1106; Chen Q, Rehman S, Smant G, Jones J T, Functional analysis of pathogenicity proteins of the potato cyst nematode Globodera rostochiensis using RNAi, Mol Plant Microbe Interact 2005; 18:621-625; Rosso M N, Dubrana M P, Cimbolini N, Jaubert S, Abad P, Application of RNA interference to root-knot nematode genes encoding esophageal gland proteins, Mol Plant Microbe Interact 2005; 18:615-620). Soaking eggs in the dsRNA solution has also been reported to induce RNAi (Fanelli E, Di Vito M, Jones J T, De Giorgi C, Analysis of chitin synthase function in a plant parasitic nematode, Meloidogyne artiellia, using RNAi, Gene 2005; 349:87-95). While these approaches are useful to study gene function in nematodes, they cannot be easily adapted as methods to increase resistance in large number of plants eg. in field crops. In addition, probably due to inefficient uptake of the dsRNA, the near null phenotype observed in C. elegans genes targeted by RNAi has not been observed in parasitic nematodes.

Approaches for inducing RNA-mediated resistance in pathogens infecting host plants are provided in the following US Patent Applications: US 20030150017, US 20040098761 and US 20060080749.

### SUMMARY OF THE INVENTION

The disclosure provides compositions and methods for modulating the expression of a target gene in pathogen through the host plant. In particular the disclosure provides a method for producing transgenic plants having a nucleic acid molecule of a pathogen, whereby the transgenic plant exhibits increased resistance to infection by the pathogen. The nucleic acid molecule of the pathogen is operably linked to sequences that allow transcription.

In accordance with one aspect, the present disclosure provides a transgenic plant having a nucleic acid molecule of a pathogen, whereby the transgenic plant has increased resistance to infection by the pathogen. The nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) a combination of (a) and (b) further comprising a spacer nucleotide sequence. The protein of the pathogen is selected from a group consisting of a protein involved in chromatin remodeling in the pathogen, a protein in a recombination, pathway of the pathogen, a protein in a nucleotide repair pathway of the pathogen, a protein for post transcriptional processing of RNA in the pathogen, and combinations thereof. In some embodiments, the protein of the pathogen is selected from a group consisting of an integrase, a splicing factor, and combinations thereof.

Further disclosure provides methods for producing transgenic plants, wherein the transgenic plant has increased resistance to infection by a pathogen, wherein the method includes transforming a plurality of first plants with a nucleic acid molecule of the pathogen to produce a plurality of second plants; and selecting the transgenic plant from the plurality of second plants, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is a combination of (a) and (b) further including a spacer nucleotide sequence, and wherein the protein of the pathogen is selected from a group consisting of a protein in a recombination pathway of the pathogen, a protein in a nucleotide repair pathway of the pathogen, a protein for post transcriptional processing of RNA in the pathogen, and combinations thereof.

In some embodiments the nucleic acid molecule of the pathogen is a double stranded (ds) DNA. In some embodiments the nucleic acid molecule of the pathogen is a ds RNA. In some embodiments the nucleic acid molecule of the pathogen is a single stranded (ss) RNA. In some embodiments the nucleic acid molecule of the pathogen is a portion of an integrase gene of the pathogen. In some embodiments the nucleic acid molecule of the pathogen is a splicing factor gene of the pathogen. In some embodiments the pathogen is a nematode. In some embodiments the nematode is Meloidogyne incognita.

In a still another aspect, the disclosure provides a recombinant vector including a nucleic acid molecule of a pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is (c) further including a spacer nucleotide sequence, and wherein the protein of the pathogen is selected from a group consisting of a protein in a recombination pathway of the pathogen, a protein in a nucleotide repair pathway of the pathogen, a protein for post transcriptional processing of RNA in the pathogen, and combinations thereof.

Another aspect provides an intermediary host cell comprising a recombinant vector, wherein the recombinant vector comprises a nucleic acid molecule of a pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is (c) further including a spacer nucleotide sequence, and wherein the protein of the pathogen is selected from a group consisting of a protein in a recombination pathway of the pathogen, a protein in a nucleotide repair pathway of the pathogen, a protein for post transcriptional processing of RNA in the pathogen, and combinations thereof. In some embodiments, the intermediary host is a bacterium. In some embodiments the intermediary host is a nematode. In some embodiments the intermediary host is E. coli. In some embodiments the intermediary host is an Agrobacterium tumefaciens.

In a still further aspect, the disclosure provides an isolated nucleic acid sequence having a nucleic acid molecule of a pathogen, wherein the nucleic acid of the pathogen is substantially homologous to a combination of (a) and (b), wherein (a) is a portion of a polynucleotide coding for a protein of the pathogen, and (b) is a sequence complementary to (a), and wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

In a further aspect, the disclosure provides a recombinant pathogen having a nucleic acid molecule of the pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is (c) further having a spacer nucleotide sequence, and wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

The disclosure provides a recombinant nematode having a nucleic acid molecule of the nematode, wherein the nucleic acid molecule of the nematode is substantially homologous to a member selected from the group consisting of (a) a portion of a polynucleotide coding for a protein of the nematode; (b) a complementary sequence to (a); (c) a combination of (a) and (b); and (d) is (c) further including a spacer nucleotide sequence, and wherein the protein of the nematode is selected from a group consisting of a protein involved in chromatin remodeling in the pathogen, a protein in a recombination pathway of the nematode, a protein in a nucleotide repair pathway of the nematode, a protein for post transcriptional processing of RNA in the nematode, and combinations thereof.

### BRIEF DESCRIPTION OF THE DRAWINGS

FIG. 1A shows roots of a typical control tobacco plant 45 days after inoculation with 2500 M incognita juveniles. The arrows point to knot-like structures formed due to infection by the parasite. The scale bar is 1 cm, except for the inset where the scale bar is 2 mm.

FIG. 1B shows roots of a transgenic plant 45 days after inoculation with 2500 M incognita juveniles. The arrows point to knot-like structures formed due to infection by the parasite. The scale bar is 1 cm, except for the inset where the scale bar is 2 mm.

FIG. 1C shows female nematodes isolated from the roots of a control tobacco plant. The scale bar is 300 μm.

FIG. 1D shows female nematodes isolated from roots of a transgenic tobacco plant. The scale bar is 300 μm.

FIG. 2 shows PCR analysis of transgenic plants transformed with a M. incognita integrase dsRNA expression cassettes. Lanes 1-8 show results of PCR amplification using PCR primers having the sequence provided in SEQ ID NOs: 19 and 32 for detecting integrase inserted in the sense orientation. Lanes 9-16 show results of PCR amplification using PCR primers having the sequence provided in SEQ ID NOs: 21 and 31 for detecting integrase inserted in the antisense orientation. Lane M provides DNA molecular size markers. Arrows point to the DNA band amplified by PCR.

FIG. 3 shows PCR analysis of transgenic plants transformed with a M. incognita splicing factor dsRNA expression cassette. Lanes 1-8 show results of PCR amplification for detecting splicing factor inserted in the sense orientation using PCR primers having the sequence provided in SEQ ID NOs: 15 and 32. Lanes 9-16 show results of PCR amplification for detecting splicing factor inserted in the antisense orientation using PCR primers having the sequence provided in SEQ ID NOs: 17 and 31. Lane M provides DNA molecular size markers. Arrows point to the DNA band amplified by PCR.

FIG. 4 shows RT-PCR amplification of actin (Genbank Accession No. CF099470), splicing factor (S) and integrase (I) cDNAs from worms isolated from control plants and transgenic plants. Lanes labeled “S.F.RNAi” show RT-PCR of total RNA from worms that were isolated from transgenic plants, which were transformed with a splicing factor dsRNA expression cassette. Lanes labeled “Integrase RNAi” show RT-PCR of worms isolated from transgenic plants, which were transformed with integrase dsRNA expression cassette. The number of PCR cycles (25, 35, 40 or 45) is indicated above the lanes. Lane M provides DNA molecular size markers.

FIG. 5 provides a recombinant vector construct having a Meloidogyne incognita integrase dsRNA expression cassette.

FIG. 6 provides a recombinant vector construct having a Meloidogyne incognita splicing factor (SF) dsRNA expression cassette.

FIG. 7A shows a single strand sequence in a Meloidogyne incognita integrase gene dsRNA expression cassette. The sequence of the sense strand of integrase gene, the spacer nucleotide sequence (intron of Arabidopsis MADS-box) and sequence of the antisense strand of integrase gene are provided. Restriction sites for Bam HI, Xho I, Kpn I and Sac I are underlined.

FIG. 7B provides a line diagram of the double stranded Meloidogyne incognita integrase gene dsRNA expression cassette in FIG. 6A. Restriction sites for Bam HI, Xho I, Kpn I and Sac I are marked.

FIG. 8A shows the sequence of the sense, intron and antisense sequence in a Meloidogyne incognita splicing factor dsRNA expression cassette. The sequence of the sense strand of splicing, factor gene, the spacer nucleotide sequence (intron of Arabidopsis MADS-box) and sequence of the antisense strand of splicing factor gene are provided. Location of restriction sites for Bam HI, Xho I, Kpn I and Sac I are underlined.

FIG. 8B provides a line diagram of the Meloidogyne incognita splicing factor dsRNA expression cassette in FIG. 7A. Restriction sites for Bam HI, Xho I, Kpn I and Sac I are indicated.

### DETAILED DESCRIPTION OF THE INVENTION

We have developed methods to produce genetically engineered plants that have increased resistance to infection by a pathogen. The methodology is based on delivering a parasite's dsRNA via a host. Most hosts for pathogens possess the basic RNA interference (RNAi) machinery and can elicit RNAi response when challenged with dsRNA. For example, the intermediate steps of RNAi mechanism include the cleavage of dsRNA into smaller fragments called short interfering RNA (siRNA) (Hamilton A J, Baulcombe D C, A species of small antisense RNA in posttranscriptional gene silencing in plants, Science 1999; 286:950-952) and, in some species, amplification of the original signal (Fire A., RNA-triggered gene silencing, Trends Genet 1999; 15:358-63). We reasoned that the production of pathogen's dsRNA in a host infected by the pathogen will induce host's RNAi machinery thereby resulting in amplification of the original RNAi signal and production of siRNA in the host. We further reasoned that the production of siRNA of the pathogen's gene in host will more readily deliver it to the pathogen during infection, which will trigger RNAi in the pathogen. Most importantly, we reasoned that this will prevent pathogen multiplication, thereby resulting in increased resistance of the host to infection by the pathogen.

We have successfully tested this idea experimentally using tobacco as the host plant and the parasitic nematode Meloidogyne incognita as the parasite and find that the dsRNA delivered through the host not only depletes the target mRNA in the parasite, but also provides the host effective resistance against the parasite (Yadav B C, Veluthambi K and Subramanian K, Host generated double stranded RNA induces RNAi in plant parasitic nematodes and protects the host from infection, Molecular and Biochemical Parasitology, 148:219-222, Apr. 19, 2006, and this document is hereby incorporated by reference in its entirety).

In the context of the present disclosure, the term “modulation” means either an increase (stimulation) or a decrease (inhibition) in the expression of a gene. In the context of the present disclosure, inhibition is the preferred form of the modulation of gene expression in a pathogen and mRNA is a preferred target in the pathogen.

As used herein, the term “plant” includes a whole plant, parts of a plant, a plant organ, a leaf, stem, root, a cell of the plant, an organelle within a cell of the plant.

A “transgenic plant” is a plant whose genome has been altered by the introduction of at least one nucleic acid molecule of a pathogen. As used herein, the term “transgenic plant” includes a whole plant, parts of a plant, a plant organ, a leaf, stem, root, a cell of the plant, an organelle within a cell of the transgenic plant.

An “explant” refers to plant cells, protoplasts, calli, roots, tubers, stems, leaves, seedlings, embryos, pollen or any plant parts or tissues, which are used for transforming a plant with a nucleic acid of pathogen.

As used herein the term “substantially homologous” refers to the extent of identity or similarity between the compared sequences. As used herein, in the context of nucleic acid sequences “substantially homologous” refers to about 75% to about 95% identity or similarity between the compared sequences. In some embodiments, substantially homologous refers to about 75%, about 80%, about 85%, about 90% or about 95% homology. In some instances, the compared nucleic acid sequences are at least 95% homologous over 50. contiguous nucleotides and have less than 95% homology in other regions.

As used herein the term “a” refers to one or more than one.

As used herein the term “about” refers to 10% more or less than the specified number.

“Nucleic acid,” “nucleic acid molecule,” or a “polynucleotide” or a “portion of a polynucleotide” as used herein refers to any DNA or RNA or a RNA/DNA hybrid molecule, either single or double stranded, and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction.

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” may refer to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The term “operably linked” refers to a nucleic acid sequence in a functional relationship with another nucleic acid sequence. For example, DNA of regulatory elements such as promoters or termination factors is operably linked to a nucleic acid sequence if the regulatory element affects the replication or transcription of the sequence. In general, “operably linked” refers to linked and contiguous DNA sequences. The sequences can be linked by ligating at convenient restriction sites. Such restriction sites may already exist on the sequences or can be introduced by synthetic oligonucleotide adaptors or linkers.

The term “primer” as used herein refers to a DNA oligonucleotide, either single stranded or double stranded, either derived from a biological system, generated by restriction enzyme digestion, or produced synthetically.

The phrase “double stranded RNA (or dsRNA) “refers to RNA with two complementary strands, similar to the DNA found in all “higher” cells. dsRNA forms the genetic material of some viruses. In eukaryotes, it acts as a trigger to initiate the process of RNA interference.

The phrase “small, interfering RNA (siRNA)” refers to a short (typically less than 30 nucleotides long) double stranded RNA molecule. Typically, the siRNA disrupts the expression of a gene to which the siRNA is targeted. Usually siRNA molecules are formed as intermediates during RNAi response. These molecules may be artificially synthesized and introduced into an organism to induce RNAi response.

The term “transformation” refers to the introduction of nucleic acid into a recipient host.

The term “host” refers to bacteria, fungi, animals or animal cells, plants or seeds, or any plant parts or tissues including plant cells, protoplasts, calli, roots, tubers, seeds, stems, leaves, seedlings, embryos, and pollen. The host is any organism or part thereof, capable of harboring the disclosed nucleic acid sequences or molecules. The host is also any organism or part thereof, capable of replicating and/or transcribing and/or translating the disclosed nucleic acid sequences. In some instances host organism is also referred to as an “intermediary host.”

“RNA transcript” refers to the product resulting from RNA polymerase-catalyzed transcription of a DNA sequence. Immediately upon transcription, it is referred to as the primary transcript. Following posttranscriptional processing of the primary transcript, it is referred to as the mature RNA. “Messenger RNA” (mRNA) refers to the RNA that is without introns and that can be translated into polypeptide by the cell. “Sense RNA” is an RNA transcript and can include a portion of an mRNA that can be translated into a polypeptide by the cell. “Antisense RNA” refers to an RNA transcript that is complementary to a sense RNA. The formation of antisense RNA in a cell can result in specific RNA:RNA duplexes being formed by base pairing between the antisense RNA substrate and a target sense RNA. The disclosure provides sense and antisense RNAs that are intracellularly expressed in a host cell from DNAs transcribed in the sense or in the antisense orientations by the host.

The disclosed nucleic acid molecules may be integrated into a chromosome in the host, or present in a recombinant vector that is not integrated into the genome, or maybe present as a transcript within the host or form disintegrated nucleic acid molecules, eg. siRNA or microRNA in the host.

As used herein, the term “portion of a protein” refers to a peptide or a polypeptide. Peptide refers to a molecule having more than one and upto 10 amino acids. A polypeptide has more than 10 amino acids and can include repeats of the peptide. As used herein a portion of a protein is a deduced amino acid sequence of a translation product of a target nucleic acid sequence.

RNAi is a mechanism for RNA-mediated regulation of gene expression in which double-stranded ribonucleic acid are known to inhibit the expression of genes with complementary nucleotide sequences.

We reasoned that plants might resist or exhibit increased resistance to infection by a pathogen is by a RNA interference (RNAi) based strategy when host plants would carry a transcribable portion of a nucleic acid sequence of a pathogen

We reasoned there would be advantages to inducing RNAi in a pathogen by delivering the pathogen's dsRNA in the form of a dsRNA expression cassette through a host for the pathogen. Most hosts possess the basic RNAi machinery and can elicit RNAi in response to dsRNA. Moreover, delivery of a nucleic acid of the pathogen by the host may provide an additional advantage of utilizing the host's RNAi machinery and thereby induce more effective RNAi in the pathogen. Intermediate steps of RNAi mechanism are known to include the cleavage of dsRNA into smaller fragments called short interfering RNA (siRNA) (Hamilton A J, Baulcombe D C, A species of small antisense RNA in posttranscriptional gene silencing in plants., Science 1999; 286:950-952) and, in some species, amplification of the original signal (A. Fire, RNA-triggered gene silencing, Trends Genet 1999; 15:358-63). We reasoned that the host plant's RNAi machinery might also produce siRNA. The host produced siRNA could be small enough to easily pass through the feeding tube used by many parasitic nematodes for nutrient uptake. Most importantly, this approach of modifying the host plant to provide a nucleic acid of the pathogen may directly serve as a tool to control parasite/pathogen infection. In addition, the host-mediated delivery of nucleic acid molecules to a pathogen could also form the basis for methods to prepare recombinant pathogens.

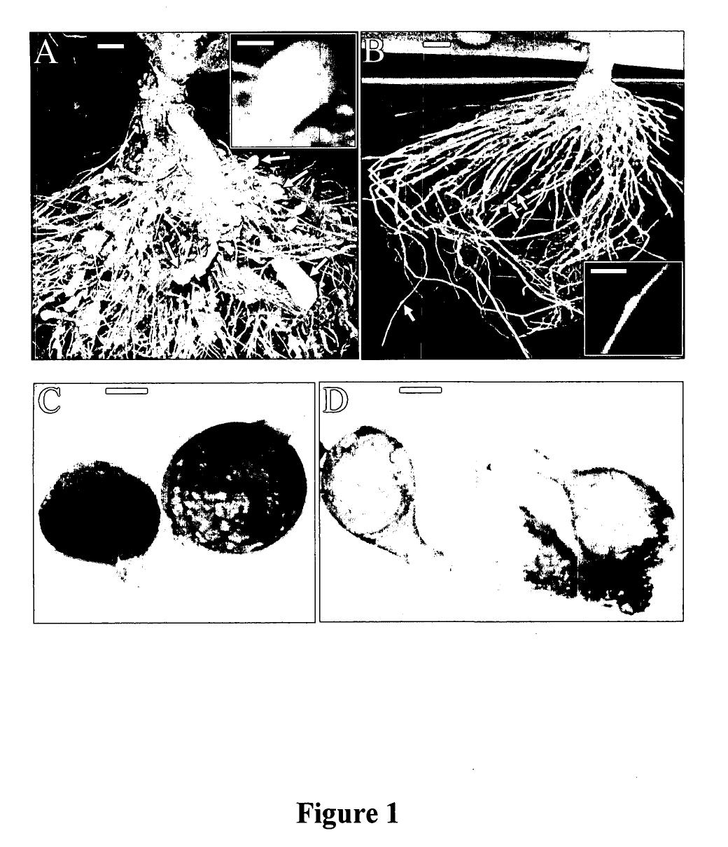

We tested host-mediated delivery of nucleic acid molecules to a pathogen experimentally using tobacco as the host plant and the parasitic nematode Meloidogyne incognita as the parasite. We found that a dsRNA expression cassette carrying a nucleic acid sequence of the pathogen when delivered through the host plant not only depleted target mRNA in the parasite, but also provided the host effective resistance against the parasite (FIGS. 1-4, Table 1, Example 13). Based on this, we developed methods for genetically engineering plants having pathogen/nematode resistance.

The target genes for RNAi were selected based on the following criteria: a) The C. elegans orthologs of the target genes in the pathogen should be essential genes and RNAi for the selected target genes should work robustly in C. elegans; b) The functions of the selected target genes of the pathogen should be conserved in diverse organisms, so the chance that their functions have been conserved in the parasitic nematodes is very high, c) The sequences of the selected target genes of the pathogen should be dissimilar enough that the RNAi is pathogen-specific.

Based on the above target gene selection criteria, in one embodiment, the present disclosure provides a transgenic plant having a nucleic acid molecule of a pathogen, whereby the transgenic plant has increased resistance to infection by the pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) a combination of (a) and (b) further including a spacer nucleotide sequence; and, wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

In some embodiments, the disclosure provides a dsRNA expression cassette having a nucleic acid sequence of the pathogen under the control of a transcriptional promoter, and expresses both a sense strand sequence and an antisense strand of a target gene of a pathogen such that the resultant transcript can form a hair-pin shaped structure. In some embodiments, the dsRNA expression cassette expresses only the sense strand sequence of the target gene of the pathogen. In some embodiments, the dsRNA expression cassette expresses only the antisense strand sequence of the target gene of the pathogen. In some embodiments, the dsRNA expression cassette expresses both the sense strand sequence and antisense sequence of the target gene of the pathogen and a spacer nucleotide sequence.

In some embodiments the nucleic acid molecule of the pathogen is a portion of a polynucleotide coding for a protein involved in a nucleotide repair pathway in the pathogen. In still yet another embodiment the present disclosure provides the transgenic plant comprising a nucleic acid molecule of a pathogen, wherein the protein involved in the recombination pathway of the pathogen is an integrase.

In another embodiment, the present disclosure provides a transgenic plant having a nucleic acid molecule of a pathogen, wherein the nucleic acid molecule codes for portion of a protein in the nucleotide repair pathway of the pathogen, and wherein the protein is an integrase. In some embodiments, the nucleic acid molecule is the antisense portion of the sequence coding for the integrase. In some embodiments the nucleic acid molecule has both the sense and antisense portion of the polynucleotide sequence coding for the integrase. In some embodiments, the nucleic acid molecule having the sense or antisense sequence of the integrase further includes a spacer nucleotide sequence.

In some embodiments the nucleic acid molecule of the pathogen is a portion of a polynucleotide coding for a protein involved in chromatin remodeling in the pathogen. In a still another embodiment, the present disclosure provides the transgenic plant having a nucleic acid molecule of a pathogen, wherein the nucleic acid of the pathogen is a portion of a polynucleotide coding for a protein involved in chromatin remodeling in the pathogen, and wherein the protein involved in chromatin remodeling in the pathogen is an integrase. In some embodiments, the integrase is from a plant pathogenic nematode M. incognita.

The integrase of M. incognita is similar to a yeast integrase called SFH1. The yeast integrase protein is known to be a component of a chromatin-remodeling complex (Cao Y et al., (1997) Sfh1p, a component of a novel chromatin-remodeling complex, is required for cell cycle progression. Mol Cell Biol 17(6):3323-3334).

In some embodiments, the nucleic acid molecule of the pathogen includes a portion of a polynucleotide coding for a protein involved in post transcriptional processing of RNA in a pathogen. (Spikes D A, Kramer J M, Bingham P M, Van Doren K, (1994), SWAP pre-mRNA splicing regulators are a novel, ancient protein family sharing a highly conserved sequence motif with the prp21 family of constitutive splicing proteins. Nucleic Acids Research 22:4510-4519). In some embodiments, the protein involved in post transcriptional processing of RNA is a splicing factor of a pathogen. In some embodiments, the protein involved in post transcriptional processing of RNA is a splicing factor of a nematode. In some embodiments, the protein involved in post transcriptional processing of RNA is a splicing factor of Meloidogyne incognita.

In some embodiments, the polynucleotide is DNA. In some embodiments the polynucleotide is double stranded DNA. In some embodiments, the polynucleotide is single stranded DNA. In some embodiments, the polynucleotide is part of a dsRNA expression cassette. In another embodiment, the polynucleotide is RNA. In still another embodiment, the polynucleotide is a double stranded RNA.

In some embodiments, the portion of the polynucleotide coding for a protein of the pathogen is a combination of a protein involved in chromatin remodeling in the pathogen and a protein in a nucleotide repair pathway of the pathogen. In some embodiments, the portion of the polynucleotide coding for a protein of the pathogen is a combination of a protein involved in chromatin remodeling in the pathogen and a protein for post transcriptional processing of RNA in the pathogen. In some embodiments, the portion of the polynucleotide coding for a protein of the pathogen is a combination of a protein involved in a protein in a nucleotide repair pathway of the pathogen and a protein for post transcriptional processing of RNA in the pathogen. In some embodiments, the portion of the polynucleotide that codes for a protein of the pathogen is a combination of a sequence of an integrase and a sequence of a splicing factor of the pathogen.

In some embodiments, the length of the portion of the polynucleotide coding for a protein of the pathogen is at least 11 nucleotides long. In some embodiments, the length of the polynucleotide is at least 21 nucleotides. In yet another embodiment, the length of the polynucleotide is at least 42 nucleotides. In still yet another embodiment, the length of the polynucleotide is at least 50 nucleotides. In some embodiments, the length of the polynucleotide is about 624 nucleotides. In some embodiments, the length of the polynucleotide is about 1238 nucleotides. In some embodiments, the length of the polynucleotide is about 349 nucleotides. In some embodiments, the length of the polynucleotide is about 963 nucleotides. In some embodiments, the length of the polynucleotide is about 1868 bp. In some embodiments, the length of the polynucleotide is about 1318 bp.

In some embodiments, the polynucleotide includes a portion of the integrase gene of Meloidogyne incognita. In some embodiments, the polynucleotide includes a portion of the splicing factor gene of Meloidogyne incognita.

In some embodiments, the polynucleotide is a member selected from the group consisting of SEQ ID NO: 1, SEQ ID NO: 2, SEQ ID NO: 27, SEQ ID NO: 28, SEQ ID NO: 29, SEQ ID NO: 30 and combinations thereof.

In some embodiments, the nucleic acid molecule is substantially homologous to a member selected from the group consisting of SEQ ID NO: 1, SEQ ID NO: 27, SEQ ID NO: 2, SEQ ID NO: 28, SEQ ID NO: 29, SEQ ID NO: 30 and combinations thereof.

SEQ ID NO: 1 includes a portion of a polynucleotide coding for a portion of an integrase protein.

SEQ ID NO: 27 includes (a) a portion of a polynucleotide coding for a portion of an integrase protein; (b) a complementary strand to (a); (c) a combination of (a) and (b); (d) a combination (a) and (b) further including a spacer nucleotide sequence.

SEQ ID NO: 2 includes a portion of a polynucleotide coding for a portion of a splicing factor protein.

SEQ ID NO: 28 includes (i) a portion of a polynucleotide coding for a portion of a splicing factor protein; (ii) a complementary strand to (i); (iii) a combination of (i) and (ii); and (iv) a combination of (i) and (ii) further including a spacer nucleotide sequence.

SEQ ID NO: 29 includes an antisense orientation of a portion of a polynucleotide coding for an integrase protein.

SEQ ID NO: 30 includes an antisense orientation of a portion of a polynucleotide coding for a splicing factor protein.

In some embodiments, the polynucleotide is selected from the group consisting of (i) a portion of a polynucleotide coding for a portion of an integrase protein; (ii) a complementary strand to (i); (iii) a combination of (i) and (ii); (iii) is a combination of (i) and (ii) further comprising a spacer nucleotide sequence; (v) a portion of a polynucleotide coding for a portion of a splicing factor protein; (vi) a complementary strand to (v); (vii) a combination of (v) and (vi); (viii) is (vii) further comprising a spacer nucleotide sequence; and, combinations thereof. In some embodiments, the deduced protein sequence is that of the integrase protein of Meloidogyne incognita. In some embodiments, the deduced protein is that of the splicing factor of Meloidogyne incognita. In some embodiments, the protein is a combination of the integrase and the splicing factor of Meloidogyne incognita. In some embodiments, the portion of the integrase protein is SEQ ID NO: 3. In some embodiments, the portion of the splicing factor protein is SEQ ID NO: 4.

In some embodiments, the polynucleotide includes a spacer nucleotide sequence. Lengths of the spacer include 10 nucleotides to about 2000 nucleotides In some embodiments the length of the spacer nucleotide sequence is about 596 nucleotides. In some embodiments, the spacer nucleotide sequence is an intron from the Arabidopsis MADS-box gene (Genbank Accession No. Y12776). In some embodiments, the spacer nucleotide sequence is an RNA molecule. In some embodiments, the spacer nucleotide is a RNA molecule and provides a structure selected from the group consisting of a hairpin, a loop or a bubble, to the dsRNA expression cassette.

In some embodiments, the present invention further provides the transgenic plant comprising a nucleic acid molecule of a pathogen. In some embodiments, the nucleic acid molecule is substantially homologous to a portion of a polynucleotide from a coding sequence for a portion of a protein of the pathogen.

The disclosure provides a method of producing transgenic plants showing increased resistance or tolerance to infection by a nematode.

In one embodiment, the present disclosure provides a transgenic plant having a nucleic acid molecule of a pathogen, whereby the transgenic plant has increased resistance to infection by the pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) a combination of (a) and (b) further including a spacer nucleotide sequence; and wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof. In some embodiments, the pathogen is a nematode. In some embodiments, the protein is an integrase, a splicing factor, or combinations thereof.

In some embodiments, the transgenic plant is a monocotyledonous plant.

In some embodiments, the transgenic plant is a dicotyledonous plant.

Plants that are affected by nematodes are distributed worldwide and include nearly every species of higher plants. (Perry, R N and Moens, M (editors), Plant Nematology, published by CABI Oxfordshire, UK, ISBN:1-84593-056-8 (2006); Luc, M, Sikora, R A and Bridge, J (editors), Plant Parasitic Nematodes in Subtropical and Tropical Agriculture, published by CABI, Oxfordshire, UK, ISBN: 0-85199-727-9 (2005)). Affected plants include vegetables, legumes, cereals, plantation crops, fruits, cash crops, tubers, spices etc. In some embodiments, the plant selected for preparing the transgenic plant is selected from the group consisting of tomato, egg plant (brinjal), chilli (green pepper), avocado, cotton, corn, pulses, peanut (ground nut), soybean, pigeon pea, chick pea, field pea, rice, wheat, oat, barley, rye, sorghum, millet, coffee, cocoa, tea, citrus, date palm, banana, pineapple, pomegranate, persimmon, papaya, mango, guava, kiwi, cotton, tobacco, sugarcane, potato, carrot, yam, taro, sweet potato, cassava, black pepper, turmeric, cardamom, lettuce, apple, plum, citrus, strawberry; ornamental trees, shrubs, Bermuda grass, azaleas, holly, conifers, sugarcane, bean, pine, oak, sycamore, palm trees, yam, nut crops, fig, mulberry, onion, sugarbeet, walnut, spruce and combinations thereof.

In some embodiments, the plant is a tobacco plant.

The present disclosure also provides a transgenic seed produced by a transgenic plant having a nucleic acid molecule of a pathogen.

The present disclosure also provides a transgenic progeny of a transgenic plant having a nucleic acid molecule of a pathogen.

In some embodiments, the pathogen is a nematode.

When a plant is infected by a pathogen, the plant exhibits a disease defense response, which is exhibited as a change in metabolism or biosynthetic activity or gene expression of the plant. The change in the infected plant enhances the plant's ability to suppress the replication and spread of a microbial pathogen whereby the plant can resist the pathogen. Agents that induce disease defense responses in plants include, but are not limited to: (1) microbial pathogens, such as fungi, oomycetes, bacteria and viruses; (2) microbial components and other defense response elicitors, such as proteins and protein fragments, small peptides, beta-glucans, eliciting, harpins and oligosaccharides; and (3) secondary defense signaling molecules produced by the plant, such as SA, H₂O₂, ethylene and jasmonates.

In some embodiments, the plant is a host for a pathogen belonging to the family selected from the group consisting of Tylenchoidea, Longidoridae and Trichodorideae.

In some embodiments, the plant is a host for a pathogen of the genus Meloidogyne, Globodera, Heterodera, Pratylenchus and Radopholus.

In some embodiments, the plant is a host for a species of the pathogen of the genus Meloidogyne selected from the group consisting of M. incognita, M. hapta, M. javanica, M. chitwoodi, M. arenaria, M. nassi, M. graminicola, M. fallax and M. artiella. Plants known to be hosts for pathogens of the genus Meloidogyne include tomato, egg plant (brinjal), chilli (green pepper), avocado, cotton, corn, pulses, peanut (ground nut), soybean, pigeon pea, chick pea, field pea, rice, wheat, oat, barley, rye, sorghum, millet, coffee, cocoa, tea, citrus, date palm, banana, pineapple, pomegranate, persimmon, papaya, mango, guava, kiwi, cotton, tobacco, sugarcane, potato, carrot, yam, taro, sweet potato, cassava, black pepper, turmeric, cardamom, etc.

In some embodiments, the plant is a host for the pathogen M. incognita.

In some embodiments, the infection in a host plant is caused directly or indirectly by Meloidogyne incognita.

In some embodiments, the plant is a host for a species of the pathogen of genus Pratylenchus selected from the group consisting of P. coffeae, P. loosi, P. brachyurus, P. neglectus, P. scribnari, P. penetrans, P. fallax, P. thornei, P. zeae, P. crenatus, P. vulnus, and P. goodeyi. Plants known to be hosts for pathogens of the genus Pratylenchus include banana, plantain, coffee, citrus, yam, tea, groundnut, potato, pineapple, peach, soybean, tobacco, rubber, cereals, turf, crucifers, legumes, strawberry, mint, maize, bean, ornamentals, fruit trees, conifers, vegetables, forage crops, sugarbeet, fern, strawberry, forage grasses, carrots, deciduous fruit and nut, citrus seedlings etc.

In some embodiments, the plant is a host for a species of the pathogen of the genus Heterodera selected from the group consisting of H. avenae, H. filipjevi, H. cajani, H. crucifererae, H. glycines, H. goettingiana, H. oryzicola, H. sacchari, H. schachtii, H. sorghi, H. trifolii, and H. zeae. Plants known to be hosts for pathogens of genus Heterodera include wheat, barley, oats, cowpea, pea, Phaseolus bean, pigeon pea, sesame, soybean, sweet corn, Brussels sprouts, broccoli, cabbage, cauliflower, kale, kohlrabi, pea, rape, rutabaga, turnip, ornamental plants, adzuki bean, broad bean, French bean, hyacinth bean, kidney bean, moth bean, mung bean, navy bean, rice bean, snap soybean, black gram, cowpea, sesame, white lupin, yellow lupin, chickpea, lentil, upland rice, banana, rice, sugarcane, Poaceae, beet, celery, chicory, Chinese cabbage, dill, sorghum, carnation, cucumber, gherkin, pumpkin, red clover, spinach, rhubarb, squash, white clover, sweet corn etc.

In some embodiments, the plant is a host for a species of the pathogen of the genus Globodera selected from the group consisting of G. pallida, G. rostochiensis, and G. tabacum. Plants known to be hosts for pathogens of Globodera include potato, tomato, aubergine and members of the solanaceae family.

In some embodiments, the disclosure provides an isolated nucleic acid sequence having a nucleic acid molecule of a pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) a combination of (a) and (b) further including a spacer nucleotide sequence; and, wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

Still another embodiment of the present disclosure provides an isolated nucleic acid sequence having a nucleic acid molecule of a pathogen, wherein the nucleic acid of the pathogen is substantially homologous to a combination of (a) and (b), wherein (a) is a portion of a polynucleotide coding for a protein of the pathogen, and (b) is a sequence complementary to (a), and wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

Still another embodiment of the present disclosure provides the isolated nucleic acid sequence that further comprises a spacer nucleotide sequence.

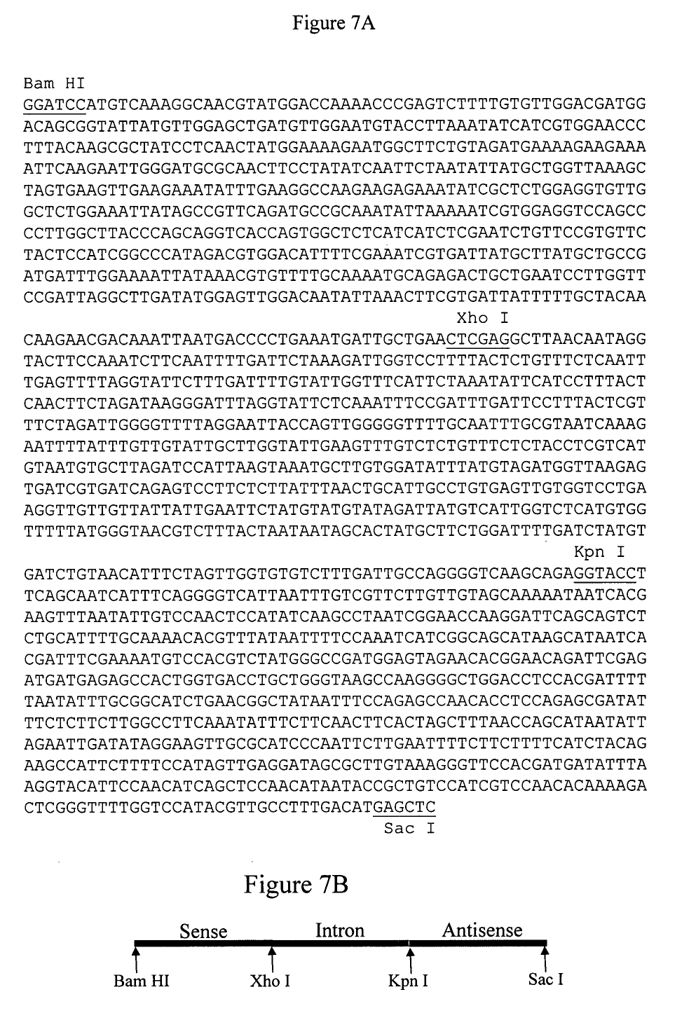

In one embodiment, the isolated nucleic acid sequence has the sequence of the sense strand of the integrase gene of Meloidogyne incognita (Genbank Accession No.: AW871671), SEQ ID NO: 1, SEQ ID NO: 27, FIG. 7A-B). In one embodiment, the isolated nucleic acid sequence has the sequence of the anti-sense strand of the integrase gene of Meloidogyne incognita (SEQ ID NO: 29). In one embodiment, the isolated nucleic acid sequence has both the sense and antisense strand sequence of the integrase gene of Meloidogyne incognita, such that transcription of the sequence can result in the formation of a single stranded RNA that folds back upon itself to form double stranded RNA. In some embodiments, the double strand RNA has a spacer nucleotide sequence. In some embodiments the spacer nucleotide sequence is from an intron of the Arabidopsis MADS-box gene (Genbank ID No. Y12776).

In one embodiment the isolated nucleic acid sequence has a portion of the polynucleotide sequence coding for the splicing factor gene of Meloidogyne incognita (Genbank Accession No. AW828516). In one embodiment, the isolated nucleic acid sequence has a portion of the polynucleotide sequence of the anti-sense strand of the splicing factor gene of Meloidogyne incognita. In one embodiment, the isolated nucleic acid sequence has both the sense and antisense strand sequence of the splicing factor gene of Meloidogyne incognita, such that transcription of the sequence can result in the formation of an RNA that can fold back upon itself to form double stranded RNA. In some embodiments, the double strand RNA can have a spacer nucleotide sequence. In some embodiments the spacer nucleotide sequence is from an intron of the Arabidopsis MADS-box gene (Genbank Accession No. Y12776).

In one embodiment the isolated nucleic acid sequence has the sequence of both the integrase (Genbank Accession No. AW871671) and the splicing factor gene of Meloidogyne incognita (Genbank Accession No. AW828516). In one embodiment, the isolated nucleic acid sequence has the sequence of the anti-sense strand of both the integrase and the splicing factor gene of Meloidogyne incognita. In one embodiment, the isolated nucleic acid sequence has both the sense and antisense strand sequence of the integrase and splicing factor gene of Meloidogyne incognita, such that transcription of the sequence can result in the formation of an RNA that can fold back upon itself to form double stranded RNA. In some embodiments, the double strand RNA can have a spacer nucleotide sequence. In some embodiments the spacer nucleotide sequence is an intron from the Arabidopsis MADS-box gene (Genbank Accession No. Y12776).

In some embodiments, the isolated nucleic acid molecule has at least 95% identity to a member selected from the group consisting of SEQ ID NO: 1, SEQ ID NO: 2, SEQ ID NO: 27, SEQ ID NO: 28, SEQ ID NO: 29, SEQ ID NO: 30 and combinations thereof.

The present disclosure further provides a recombinant vector having a nucleic acid molecule of a pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is (c) further having a spacer nucleotide sequence, and wherein the protein of the pathogen is selected from a group consisting of a protein in a recombination pathway of the pathogen, a protein in a nucleotide repair pathway of the pathogen, a protein for post transcriptional processing of RNA in the pathogen, and combinations thereof. For the expression of dsRNA in transgenic plants conventional plant transformation vectors eg. pBI121 and plant RNAi vector can be used to prepare a dsRNA expression cassette. In some embodiments, the recombinant vector is one of pBC16, pBC17, pBC20, or pBC21.

In some embodiments, a recombinant vector is one of pBC16, pBC17, pBC20 or pBC21. pBC16 has a dsRNA expression cassette under the control of a CaMV 35S promoter for expressing a nucleic acid of the integrase gene of Meloidogyne incognita. pBC17 has a dsRNA expression cassette under the control of root-specific promoter for expressing a nucleic acid molecule of the integrase gene of Meloidogyne incognita. pBC20 has a dsRNA expression vector under the control of a CaMV 35S promoter for expressing a nucleic acid molecule of the splicing factor gene of Meloidogyne incognita. pBC21 has a dsRNA expression cassette under the control of root-specific promoter for expressing a nucleic acid molecule of the splicing factor gene of Meloidogyne incognita.

Recombinant constructs pBC16, pBC17, pBC20, pBC21 have been deposited at Microbial Type Culture Collection and Gene Bank (MTCC) at Institute of Microbial Technology, Chandigarh, India. The deposit numbers assigned to the recombinant constructs are MTCC5346 (for construct pBC16); MTCC5347 (for construct pBC17); MTCC5348 (for construct pBC20); and MTCC5349 (for construct pBC21). Recombinant constructs pBC16, pBC17, pBC20 and pBC21 are also referred to as KS100, KS101, KS102 and KS103, respectively. In some embodiments, the recombinant construct is one of MTCC5346, MTCC5347, MTCC5348 or MTCC5349.

The recombinant vector of the present disclosure further includes the nucleic acid molecule of the pathogen operably linked to a promoter, and wherein the promoter is selected from a group consisting of a constitutive promoter, a tissue specific promoter, an inducible promoter, a wound inducible promoter, and combinations thereof. In some embodiments, the promoter is a CaMV 35S promoter (SEQ ID NO: 35; Genbank Accession No. S45406). In some embodiments, the promoter is a root-specific promoter (SEQ ID NO: 26).

Various promoters such as nos, ocs, mas, plant Actin promoters (Act-1), Adh-1, Ubiquitin promoter, alcohol-regulated, steroid-regulated, pathogenesis-related promoter, auxin-regulated can be used for the expression of the double stranded RNA of integrase and splicing factor in a plant.

The CaMV 35S promoter (pBC6 SEQ ID NO: 35) or a tobacco root-specific promoter (Yamamoto Y T, Taylor C G, Acedo G N, Cheng C L, Conkling M A. Characterization of cis-acting sequences regulating root-specific gene expression in tobacco, Plant Cell 1991; 3:371-382) (pBC7, SEQ ID NO: 26) can be placed upstream of the intron of the Arabidopsis MADS-box gene (Y12776) flanked by two multiple cloning sites.

Plant transformation vectors such as pBI121 or any other vector suitable for dsRNA expression in plants may be employed for producing the recombinant vector construct.

The recombinant vector of the present disclosure further includes at least one selectable marker. Selectable markers include nucleotide sequences that confer antibiotic resistance to an organism eg., ampicillin, kanamycin, neomycin, hygromycin resistance etc.

In another embodiment, the present disclosure provides an intermediary host cell having a recombinant vector, wherein the recombinant vector comprises a nucleic acid molecule of a pathogen, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is (c) further including a spacer nucleotide sequence, and wherein the protein of the pathogen is selected from a group consisting of a protein in chromatin remodeling of the pathogen, a protein in a recombination pathway of the pathogen, a protein in a nucleotide repair pathway of the pathogen, a protein for post transcriptional processing of RNA in the pathogen, and combinations thereof. The intermediary host can be a plant pathogen such as Agrobacterium, which can transfer a polynucleotide to a plant. In some instances the intermediary host is a bacteria such as E. coli.

In still another embodiment, the present disclosure provides a method of producing a transgenic plant, wherein the transgenic plant has increased resistance to infection by a pathogen. The method producing a transgenic plant having increased resistance to infection from a pathogen includes transforming a plurality of first plants with a nucleic acid molecule of the pathogen to produce a plurality of second plants; and selecting the transgenic plant from the plurality of second plants, wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is a combination of (a) and (b) further including a spacer nucleotide sequence, and wherein the protein of the pathogen is selected from a group consisting of a protein involved in chromatin remodeling of the pathogen, a protein in a recombination pathway of the pathogen, a protein in a nucleotide repair pathway of the pathogen, a protein for post transcriptional processing of RNA in the pathogen, and combinations thereof.

In another embodiment, the method producing a transgenic plant further includes transforming a plant with an intermediary host having the nucleic acid of the pathogen. In some embodiments the intermediary host is a bacteria. In some embodiments the intermediary host is an E. coli. In some embodiments, the intermediary host is an E. coli having a dsRNA expression cassette for the integrase gene of Meloidogyne incognita. In some embodiments, the intermediary host is an E. coli carrying a dsRNA expression cassette for the splicing factor gene of Meloidogyne incognita. In some embodiments, the intermediary host is an Agrobacterium. In some embodiments, the intermediary host is an Agrobacterium tumefaciens. In some embodiments, the intermediary host is an Agrobacterium tumefaciens having a dsRNA expression cassette for the integrase gene of Meloidogyne incognita. In some embodiments, the intermediary host is an Agrobacterium tumefaciens carrying a dsRNA expression cassette for the splicing factor gene of Meloidogyne incognita.

In one embodiment, the disclosure provides a method of producing a transgenic plant by transforming a plant with a Agrobacterium tumefaciens carrying a recombinant vector of an integrase or a splicing factor dsRNA expression cassette. The method includes constructing a recombinant vector having a nucleic acid of the pathogen and mobilizing the recombinant vector in an Agrobacterium tumefaciens cell to produce a recombinant Agrobacterium tumefaciens cell. An explant is prepared from a plant. A plurality of explants is co-cultivated with the recombinant Agrobacterium tumefaciens to produce a plurality of transformed explants. The transformed explants are cultured and grown from which a transgenic plant is selected by techniques such as PCR or bioassays confirming resistance of the transgenic plant to infection by the pathogen.

The disclosure provides methods to prepare a transgenic plant having a recombinant vector of the disclosure. The disclosure provides methods to select a transgenic plant having a recombinant vector of the disclosure. The disclosure provides a method of producing a transgenic plant having a nucleic acid molecule of a pathogen, and wherein the transgenic plant has increased resistance to infection by the pathogen. Explants are prepared by injuring leaves of a plant to form injured leaf discs. The injured leaf discs are then placed on an antibiotic containing agar-medium. A recombinant Agrobacterium carrying a nucleic acid of the pathogen is added to the injured leaf discs resulting in Agrobacterium transformed leaf discs or leaves. The Agrobacterium transformed leaves or leaf discs are grown and tested for the presence of the nucleic acid molecule of the pathogen. Tests such as RT-PCR, PCR, western blotting or southern blotting allow selection of a transgenic plant carrying the nucleic acid of the pathogen. The ability of the transgenic plant to resist infection by a pathogen, or show increased resistance to infection by a pathogen, or show tolerance to infection by a pathogen is confirmed by infecting the transgenic plant with the pathogen and the ability of the transgenic plant to respond to or to overcome infection by the pathogen.

Further, the disclosure provides a method of producing transgenic plants having nucleotide sequence of target pathogen genes for RNAi by co-cultivating host plants with recombinant Agrobacterium which carries a recombinant vector construct of dsRNA expression cassette of the target gene.

The disclosure provides methods of Agrobacterium transformation of plants. The recombinant vector construct can be introduced into an Agrobacterium tumefaciens which can be used to infect the plant cell, thereby transferring the foreign nucleotide molecule into the plant cell and conferring the pest resistance. Various Agrobacterium strains such as LBA 4404, FEH 101, EHA 105 can be used for transformation. The recombinant vector construct can be transformed into an Agrobacterium cell by methods well known in the art including electroporation or cold shock method.

Another embodiment relates to plant transformation methods. Plants can be transformed with a recombinant construct by various methods of transformation known in the art such as electroporation, particle bombardment, in planta transformation, Agrobacterium mediated transformation, seed transformation etc.

Yet another embodiment provides an explant for plant transformation. An explant can be a plant cell, a protoplast, callus tissue, a root, a tuber, a stem, a leaf, a seedling, an embryo, pollen or any plant part or tissue.

The disclosure provides a method for selection of target genes of a pathogen for inducing RNAi in the pathogen. The disclosure provides a method for selection of target genes of nematodes for inducing RNAi in the nematode. In some embodiments, the target gene for RNAi is in a nematode and codes for an integrase of M. incognita (Genbank Accession No.: AW871671, SEQ ID NO: 5), M. hapla, M. arenaria, M. javanica, M. nassi, M. graminicola, M. fallax, M. chitwoodi or M. artiella.

For example, integrase sequences from other plant pathogens, such as, SEQ ID NO: 6 (Heterodera glycines, Genbank Accession No. CB281426), SEQ ID NO: 7 (Meloidogyne paranaensis, Genbank Accession No.: CN477873), SEQ ID NO: 8 (Strongyloides ratti, Genbank Accession No.: BM880203), SEQ ID NO: 9 (Brugia malayi, Genbank Accession No.: R86426) can be selected as targets for inducing RNAi in the respective pathogens.

In some embodiments, the target gene for RNAi is in a nematode and codes for a splicing factor of M. incognita (SEQ ID NO: 10, Genbank Accession No.: AW828516), M. hapla, M. arenaria, M. javanica, M. nassi, M. graminicola, M. fallax, M. chitwoodi or M. artiella.

For example, splicing factor sequences from other plant pathogens, such as, SEQ ID NO: 11 (Heterodera glycines, Genbank Accession No.: CB825077), SEQ ID NO: 12 (Heterodera glycines, Genbank Accession No.: CB380114), SEQ ID NO: 13 (Meloidogyne hapla, Genbank Accession No.: CF979876), SEQ ID NO: 14 (Ascaris suum, Genbank Accession No.: BM283662) can be selected as targets for inducing RNAi in the respective pathogens.

Methods for selecting transgenic plants having a nucleic acid sequence of pathogen include PCR, RT-PCT, Northern blotting, Southern blotting, Western blotting etc. to detect the presence of nucleic acid of the pathogen or products of expression of the nucleic acid of the pathogen such as RNA or protein. Other methods of selection of a transgenic plant having the nucleic acid of a pathogen include testing the ability of the transgenic plant to resist infection by a pathogen, or increased resistance by the plant to infection by a pathogen. Plant infection experiments can be performed under standard green house conditions.

For example, M. incognita from a local field can be maintained on tobacco plants and eggs can be hatched on moist filter paper. Freshly hatched juveniles can be transferred to the soil close to roots of the transgenic plants. Soil can be washed off from the roots and roots can be examined for infection after several days. The nematodes can be dissected out of the roots with the help of forceps and a stereomicroscope.

The disclosure provides a method of preparing a pathogen having altered gene expression of a target gene of the pathogen. The method includes infecting transgenic plants with a pathogen. The transgenic plants carry a nucleic acid of the pathogen in the form of a dsRNA expression cassette for inducing RNAi in a target gene of the pathogen. Some of the pathogens that infect the transgenic plant become altered or modified pathogens carrying all or a part of the dsRNA of the target gene of the pathogen. In some embodiments, pathogens exhibit altered growth characteristics (See, fusiform and elongated Meloidogyne incognita in FIG. 1D). In some embodiments, the altered pathogen is one or more of M. incognita, M. hapla, M. arenaria, M. javanica, M. nassi, M. graminicola, M. fallax, M. chitwoodi or M. artiella. In some embodiments, the altered pathogen is Meloidogyne incognita. In some embodiments, the altered pathogen carries all or part of the Meloidogyne incognita integrase dsRNA for inducing RNAi against the integrase gene of Meloidogyne incognita. In some embodiments, the altered or modified pathogen carries all or part of the Meloidogyne incognita splicing factor dsRNA for inducing RNAi against the splicing factor gene of Meloidogyne incognita. The presence of the nucleic acid of the pathogen in the altered pathogen can be detected by conventional methods such as PCR, RT-PCR, northern blotting, western blotting or southern blotting etc.

While the invention is broadly as defined above, it will be appreciated by those persons skilled in the art that it is not limited thereto and that it also includes embodiments of which the following description gives examples.

It should be understood that the following examples described herein are for illustrative purposes only and that various modifications or changes in light will be suggested to persons skilled in the art and are to be included within the spirit and purview of this application and the scope of the appended claims.

### EXAMPLES

### Example 1

Isolation of RNA from Nematode

Nematodes were lysed in 10 volumes of Tri-reagent (Sigma Chemical Company, St. Louis, Mo., USA) by homogenization in 1.5-ml microfuge tube. The lysate was incubated at room temperature for 15 minutes, following which 0.2 volumes of chloroform were added, mixed and centrifuged at 8000 g for 5 minutes. The upper aqueous phase was collected in a fresh tube and the RNA was precipitated with 0.5 volumes of isopropanol. The RNA precipitate was collected as a pellet after centrifugation at 4° C. for 15 minutes at 12000 g. The RNA pellet was washed in 70% ethanol, air dried and dissolved in 50 μl of TE buffer (10 mM Tris, 1 mM EDTA), pH 7.0 and stored at −20° C.

### Example 2

RT-PCR for Isolation of Integrase Gene of M. incognita

One 624-bp fragment of integrase cDNA for cloning in a sense orientation and another 624-bp fragment of integrase cDNA for cloning in an antisense orientation were obtained from total RNA of Meloidogyne incognita by RT-PCR.

Total RNA from nematodes was extracted as described in Example 1. RT-PCR was performed using reverse transcriptase (RevertAid M-MuLV reverse transcriptase; Fermentas, Vilnius, Lithuania) under conditions suggested by the manufacturer. The single-stranded cDNA obtained by reverse transcription of nematode RNA was diluted with TE buffer, pH 7.5 and stored at −20° C. General procedures are described in Sambrook J, Russell D W, Molecular cloning: A Laboratory Manual, Cold Spring Harbor, Cold Spring Harbor Laboratory Press, 2001.

### PCR Amplification

A 624-bp fragment of the integrase cDNA (Accession No. AW871671) was amplified by PCR in sense and antisense directions using the primers having oligonucleotide sequence as set forth in SEQ ID NO: 19, 20, 21, and 22.

SEQ ID NO: 19

KS1094: TCTGGATCCATGTCAAAGGCAACGTATGGA

SEQ ID NO: 20

KS1095: TCTCTCGAGTTCAGC-AATCATTTCAGGGG

SEQ ID NO: 21

KS1096: TCTGAGCTCATGTCAAAGGCAACGTATGGA

SEQ ID NO: 22

KS1097: TCTGGTACCTTCAGCAATCATTTCAGGGG

A 624-bp fragment of the integrase cDNA (Accession No. AW871671) was amplified in sense and antisense directions from the above described single stranded cDNA from the RT-PCR reaction using the primers of SEQ ID NOs.: 19-22.

Primers having SEQ ID NO: 19 and SEQ ID NO: 20 were used to amplify the 624 bp long of integrase cDNA for cloning in a sense orientation in plant expression vectors.

Primers having SEQ ID NO: 21 and SEQ ID NO: 22 were used to amplify a 624 bp long of integrase cDNA for cloning in an antisense orientation in plant expression vectors.

PCR settings were 95° C., 2 min; 35 cycles of 15 s at 95° C., 20 s at 55° C. and 1 min at 72° C., and 10 min at 72° C. using Taq polymerase (Bangalore-Genei, Bangalore, India).

DNA sequence in the PCR amplified products in the sense strand orientation is provided as SEQ ID NO: 1. DNA sequence of PCR amplified product in the antisense strand orientation is provided as SEQ ID NO:29.

### Example 3

Isolation of Splicing Factor cDNA from M. incognita

A 349-bp fragment of splicing factor cDNA from M. incognita juveniles was amplified by conventional techniques. Total RNA extracted was from the nematodes by as described in Example 1. The reverse transcription-polymerase chain reaction (RT-PCR) was performed as described in Example 2. The single-stranded cDNA obtained by reverse transcription of nematode RNA was diluted with TE buffer, pH 7.5 and stored at −20 ° C.

### PCR Amplification:

A 349-bp splicing factor sequence (Accession No. AW828516) was amplified in sense and antisense directions using the primers of SEQ ID NOs.: 15-18.

SEQ ID NO: 15

KS1098: TCTGGATCCCTGCTCTTTTCGTTGCACGT

SEQ ID NO: 16

KS1099: TCTCTCGAGTGTGT-GAGAAATTGACGTCC

SEQ ID NO: 17

KS1100: TCTGAGCTCCTGCTCTTTTCGTTGCACGT

SEQ ID NO: 18

KS1101: TCTGGTACCTGTGTGAGAAATTGACGTCC

Primers having SEQ ID NO: 15 and SEQ ID NO: 16 were used to amplify a 349-bp long splicing factor cDNA for cloning in sense orientation in the plant expression vectors. Primers having SEQ ID NO: 17 and SEQ ID NO: 18 were used to amplify a 349 bp long splicing factor cDNA for cloning in antisense orientation in the plant expression vectors:

PCR settings were 95° C., 2 min; 35 cycles of 15 s at 95° C., 20 s at 55° C. and 1 min at 72° C., and 10 min at 72° C. using Taq polymerase (Bangalore-Genei, Bangalore, India) and the reaction was set up under conditions recommended by the manufacturer.

The two isolated DNAs were cloned in the sense and antisense orientations in suitable plasmid vectors to produce recombinant vectors. The DNA sequence for the sense orientation of the splicing factor from M. incognita is set forth in SEQ ID NO: 2. Sequence of the antisense fragment of M. incognita splicing factor DNA is set forth in SEQ ID NO: 30.

### Example 4

### Promoters and Plasmids in Recombinant Plant Vectors

The plasmid with the cauliflower mosaic virus (CaMV) 35S promoter was named pBC6. The plasmid having a root-specific promoter was named pBC7. The intron of Arabidopsis MADS box gene was amplified and cloned in the parent plasmid pBI121 (accession#485783), which carries CaMV 35S promoter (SEQ ID NO: 35), to generate pBC6. The same intron, and the root-specific promoter sequence (GenBank Accession No. S45406, SEQ ID NO: 26) amplified from tobacco genomic DNA were cloned in the parent plasmid pBI101 (GenBank Accession No. U12639) to generate pBC7.

### Example 5

### Amplification of Tobacco Root Specific Promoter

Tobacco root-specific promoter, TobRB7 (GenBank Accession No. S45406, Yamamoto Y T, Taylor C G, Acedo G N, Cheng C L, Conkling M A, Characterization of cis-acting sequences regulating root-specific gene expression in tobacco., Plant Cell 1991; 3:371-82.) was amplified from tobacco genomic DNA. The primers used to amplify the root specific promoter are

SEQ ID NO: 33

KS1001: TCTTCTAGATCCTACACAATGTGAATTTG

SEQ ID NO: 34

KS1002: TCTTCTAGATTCTCACTAGAAAAATGCCC

The nucleotide sequence of the amplified tobacco root specific promoter is shown in SEQ ID NO: 26.

### Example 6

### Intron (Spacer Nucleotide Sequence) in Recombinant Plant Vectors

### Vector Preparation

pBC6

A 596 bp long polynucleotide (see SEQ ID NO: 25), which includes a 572 bp intron sequence of the Arabidopsis MADS-box gene (GenBank accession# Y12776), was PCR-amplified from Arabidopsis genomic DNA and cloned between the Bam HI and Sac I sites of the binary vector pBI121 (accession# AF485783) to generate pBC6. It is expected that RNA processing of SEQ ID No: 25 will cause the intron in the loop to be removed while bases 1-13 and 584-596 will remain. The cloning replaced the β-glucuronidase gene in pBI121 with the MADS-box intron. To facilitate the cloning of cDNA inserts of RNAi-target genes, we introduced XhoI and KpnI sites at the 5′ and 3′ ends, respectively, of the intron through the primers used in the above PCR. The oligonucleotide sequence of the primers used to amplify the MADS-box intron and to introduce the XhoI and KpnI sites is set forth in SEQ ID NO: 23 and SEQ ID NO: 24. The sequence of the MADS-box intron is provided in SEQ ID NO: 25.

SEQ ID NO: 23

(KS1080) TCTGGATCCCTCGAGGCTTAACAATAGGTACTITCC

SEQ ID NO: 24

(KS1081) TCTGAGCTCGGTACCTCTGCTTGACCCCTGGCAAT

### Example 7

Construction of Recombinant Plant Vector Having M. incognita Integrase dsRNA Expression Cassette
Preparation of Recombinant Vector pBC16

The 596 bp spacer nucleotide sequence amplified from the Arabidopsis (Example 6) was cloned in BamHI and SacI sites of the binary vector pBI121 (accession# AF485783) to obtain a recombinant vector pBC6. To facilitate the cloning of cDNA inserts of RNAi-target genes, XhoI and KpnI sites were introduced at the 5′ and 3′ ends, respectively, of the intron through the primers used in the above PCR.

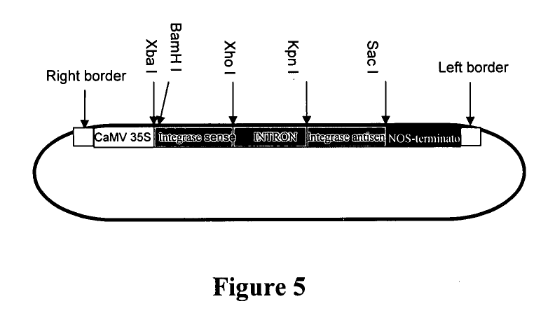

The 624-bp of cDNA (sense strand, SEQ ID NO:1) of integrase gene isolated from M. incognita (Example 2) was cloned in BamHI and XhoI sites of the recombinant vector pBC6. Further the antisense strand of SEQ ID NO: 1 was cloned in KpnI and SacI sites of the recombinant vector pBC6. Thus the β-glucuronidase gene in pBI121 was replaced with 1868-bp DNA fragment (SEQ ID NO: 27) containing the sense and antisense strand of the integrase gene intercepted with a spacer sequence. The spacer sequence is as provided in SEQ ID NO: 25, and includes the intron of Arabidopsis thaliana. The recombinant vector thus constructed was named as pBC16. Schematic map of the recombinant vector pBC16 is shown in FIGS. 5. Sequence of the dsRNA expression cassette (SEQ ID NO: 27) for the integrase gene is provided in FIGS. 7A and 7B. pBC16 is also referred to as one of the recombinant vectors carrying the integrase dsRNA expression cassette.

Preparation of Recombinant Vector pBC17:

Tobacco root-specific promoter of TobRB7 (Yamamoto Y T, Taylor C G, Acedo G N, Cheng C L, Conkling M A, Characterization of cis-acting sequences regulating root-specific gene expression in tobacco., Plant Cell 1991; 3:371-382.) (Example 5) of 706 bp was cloned in the XbaI site of the promoter-less binary vector, pBI101 (accession# AF485783). The spacer sequence amplified from Arabidopsis thaliana genomic DNA (Example 6) was cloned in the BamHI and SacI sites of pBI101 containing the tobacco root-specific promoter to obtain the recombinant vector pBC7 vector.

The 624-bp of cDNA (sense strand, SEQ ID NO: 1) of integrase gene isolated from M. incognita (Example 2) was cloned in Bam HI and Xho I sites of the recombinant vector pBC7. The 624-bp of cDNA (antisense strand) of integrase gene (SEQ ID NO: 1) was cloned in Kpn I and Sac I sites of the recombinant vector pBC7. The recombinant vector thus constructed was designated as pBC17. pBC17 contains tobacco root specific promoter (Genbank Accession No. S45406; provided herein as SEQ ID NO: 26) for driving the expression of target gene of the nematode in the root of the host plant. Sequence of the dsRNA expression cassette (SEQ ID NO: 27) for the integrase gene including the restriction sites is provided in FIGS. 7A and 7B. pBC17 is also referred to as one of the recombinant vectors carrying the integrase dsRNA expression cassette.

### Example 8

Construction of Recombinant Plant Vector Having M. incognita Splicing Factor dsRNA Expression Cassette
Preparation of pBC20

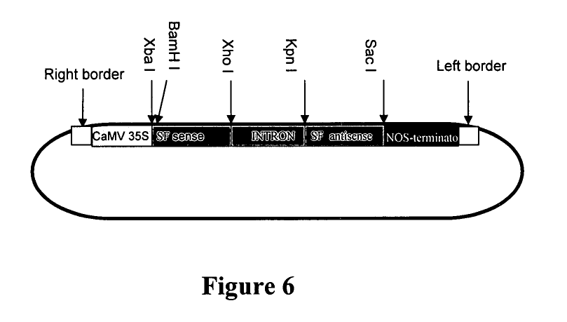

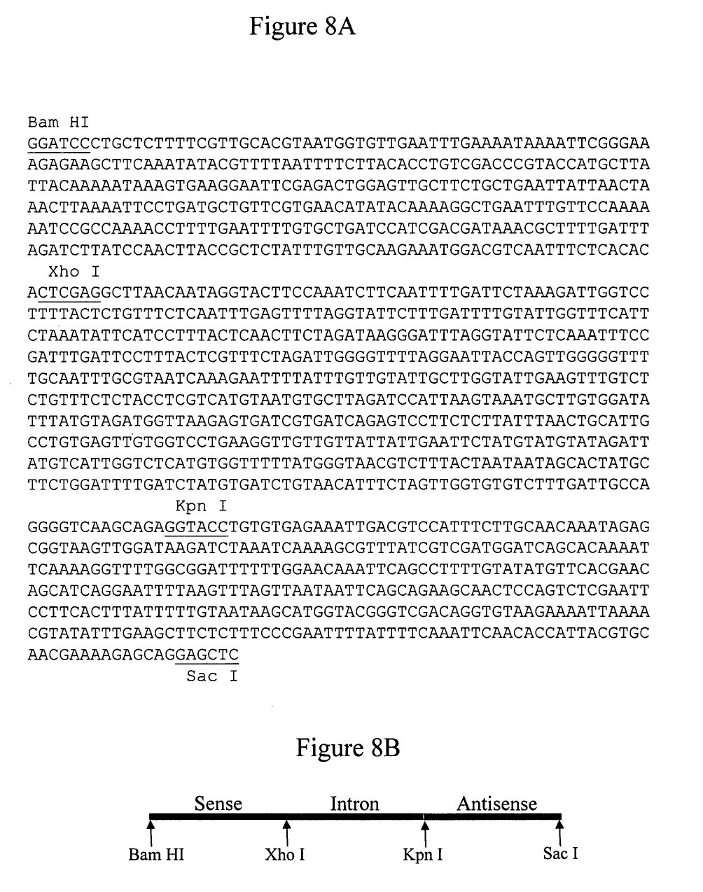

A 349-bp DNA sequence of sense strand of the gene coding for splicing factor isolated from M. incognita is as set forth in SEQ ID NO: 2. Similar to Example 7, a recombinant vector comprising the sense (349 bp) and antisense (349 bp) strand of the splicing factor of M. incognita and spacer sequence (596 bp) from Arabidopsis (Example 4) was constructed and was named as pBC20. Thus the β-glucuronidase gene in pBI121 was replaced with 1318 bp DNA fragment containing the sense and antisense strand of the splicing factor gene intercepted with a spacer sequence. The spacer sequence (SEQ ID NO: 25) includes the intron described above of Arabidopsis thaliana. Map of the recombinant vector is shown in FIG. 6. Sequence (SEQ ID NO: 28) of the dsRNA expression cassette having the sense strand of splicing factor, intron sequence and antisense strand of splicing factor and the restriction sites therein is provided in FIGS. 8A and 8B. The promoter for expression of the dsRNA cassette in pBC20 recombinant vector is the CaMV promoter. pBC20 is also referred to as one of the recombinant vectors carrying the splicing factor dsRNA expression cassette.

Preparation of pBC21

Similar to the description for cloning of pBC17, a recombinant vector having the 349-bp sense strand sequence (SEQ ID NO: 2) and 349-bp antisense strand sequence (SEQ ID NO: 30) of the splicing factor of M. incognita and 596-bp spacer sequence (SEQ ID NO: 25) from Arabidopsis under the control of a tobacco root-specific promoter was constructed and was named as pBC21. Sequence (SEQ ID NO: 28) of the dsRNA expression cassette having the sense strand of splicing factor, intron sequence and antisense strand of splicing factor and the restriction sites therein is provided in FIGS. 8A and 8B. pBC21 is also referred to as a recombinant vector carrying the splicing factor dsRNA expression cassette.

### Example 9

Transformation of Bacteria with Recombinant Vector Carrying Integrase dsRNA Expression Cassette

The recombinant vectors pBC16 and pBC17 having the dsRNA expression cassette for sense sequence of integrase were transformed into E. coli and Agrobacterium cells using methods known in the art to produce recombinant host cells.

The recombinant vectors pBC16 and pBC17 having the integrase dsRNA expression cassette were transformed into Agrobacterium tumefaciens strain LBA4404 through tri-parental mating using pRK2013 as mobilization helper (Ditta G, Stanfield S, Corbin D, Helinski D R, Broad host range DNA cloning system for gram-negative bacteria: construction of a gene bank of Rhizobium meliloti, Proc Natl Acad Sci USA 1980; 77:7347-7351).

In brief, E. coli DH5α cells harboring plasmid pBC16 or pBC17 were mixed with an equal volume of cells of E. coli strain DH5α harboring the helper plasmid pRK2013, and, with twice the volume of the Agrobacterium strain LBA4404. The mixture of E. coli and Agrobacterium cells was spread on YEP plates and allowed to grow for 12-16 hours at 28° C. The overnight culture was serially diluted up to a million times in saline solution and plated on an agar plate containing rifampicin and kanamycin. The antibiotics allowed selective growth of Agrobacterium cells that had successfully received the kanamycin encoding plasmid from the E. coli cells because Agrobacterium strain LBA4404 carried a rifampicin resistance marker on its chromosome and plasmids pBC16 or pBC17 carried neomycin phosphotransferase for kanamycin resistance.

### Example 10

Transformation of Bacteria with Recombinant Vector Carrying Splicing Factor dsRNA Construct

The recombinant vectors pBC20 and pBC21 having the splicing factor dsRNA expression sequence were transformed into E. coli and Agrobacterium cells as described in Example 9.

### Example 11

Plant Transformation with Agrobacterium

Recombinant Agrobacterium tumefaciens, which had integrase dsRNA expression construct (See Example 10), were co-cultivated with tobacco leaf discs according to methods provided in Sunilkumar G, Vijayachandra K, Veluthambi K, Preincubation of cut tobacco leaf explants promotes Agrobacterium-mediated transformation by increasing vir gene induction., Plant Science 1999; 141:51-58. At least 20 independent lines of transgenic plants (confirmed by PCR) were generated per construct.

Similarly, recombinant Agrobacterium tumefaciens carrying splicing factor dsRNA expression construct prepared as described in Example 10 were co-cultivated with tobacco leaf discs.

Agrobacterium-mediated transformation of tobacco (Nicotiana tabacum) cv. Wisconsin was carried out. Briefly, sterile tobacco leaf discs were cut and transferred to Murashige and Skoog (MS) medium containing 3% sucrose, 1 mg/L BAP, 1 mg/L NAA, 0.8% Bacto-Agar, pH 5.6 at 28° C. in 16 hours light and 8 hours darkness for 24 hours prior to transformation. 100 ml of an overnight grown culture of transformed Agrobacterium strain was resuspended in 0.5×MS liquid medium with 3% sucrose, pH 5.6 (5 ml). The leaf discs were subsequently co-cultivated with the Agrobacterium (transformed with integrase or splicing factor dsRNA expression constructs) for 30 minutes. The discs were dried on sterile No. 1 Whatmann discs and transferred to MS medium containing 3% sucrose, 1 mg/L Benzyl amino purine (BAP), 1 mg/L Napthalene acetic acid (NAA), 0.8% Bacto-Agar, and pH 5.6 at 28° C. in 16 hours light and 8 hours darkness for 48 hrs. The leaf discs were given several washes in half strength liquid MS medium with 1.5% sucrose, pH 5.6 containing 250 mg/mL cefotaxime. Excess moisture on the leaf discs was blotted on sterile Whatmann No. 1 filter paper. The discs were then placed on selection media, that is, MS medium containing 3% sucrose, 1 mg/L BAP, 1 mg/L NAA, 0.8% Bacto-Agar, pH 5.6 containing 250 mg/mL cefotaxime and 25 mg/L hygromycin at 28° C. in 16 hours light and 8 hours darkness. The leaf discs were transferred to fresh selection media every 14 days until multiple shoot regeneration was seen. Shoot regeneration was seen between 20-45 days after first placing on the selection media. Regenerated independent shoots were then transferred to rooting medium (MS medium containing 3% sucrose, 0.8% Bacto-Agar, pH 5.6 containing 250 mg/mL cefotaxime and 25 mg/L hygromycin at 28° C. in 16 hours light and 8 hours darkness). After establishment of roots in the medium, the plants that grew out were transferred to fresh rooting medium on which they were maintained for further experiments.

### Example 12

Identification of Transgenic Plants by Analysis of Plants Obtained after Co-Cultivation with Agrobacterium

Confirming presence of dsRNA expression cassette in plants by PCR

Presence of the dsRNA expression cassette in the genome of the transgenic plants was confirmed by PCR.

DNA isolation from transgenic plant:

Template used for the PCR was genomic DNA isolated from leaves using the DNeasy mini kit available from Qiagen (Qiagen GmbH, Hilden, Germany) using protocol suggested by the manufacturer. Briefly, about 100 mg of leaves were ground to fine powder in liquid nitrogen using a mortar and pestle, and used for DNA extraction.

PCR amplification

The DNA extracted from the leaves was PCR amplified using primers of SEQ ID NO: 19 and SEQ ID NO: 32; SEQ ID NO: 21 and SEQ ID NO: 31 to detect integrase. Primers of SEQ ID NO: 15 and SEQ ID NO: 32; SEQ ID NO: 17 and SEQ ID NO: 31 were used for detection of splicing factor.

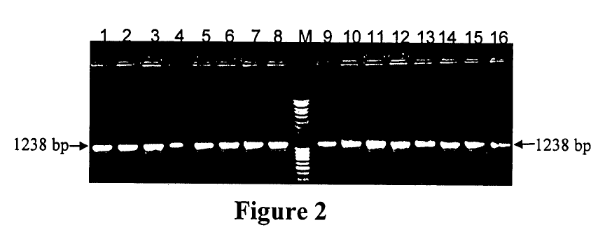

Successful amplification of DNA bands of the size predicted from SEQ ID NOS 27 and 28 would indicate the presence of the dsRNA expression cassettes in the transgenic plants and, therefore, confirm successful transformation. As shown in FIGS. 2 (integrase) and 3 (splicing factor), the predicted DNA bands were amplified from all the tested plants. Thus, these results confirm the presence of dsRNA expression constructs in these plants.

DNA from transgenic plants transformed with a M. incognita integrase dsRNA expression cassette was analyzed by PCR. Amplification using primers having sequence as provided in SEQ ID NOs: 19 and 32 resulted in a 1238 bp fragment, which includes sequences from the sense orientation of the integrase gene and the spacer sequence (FIG. 2, lanes 1-8). The detected 1238-bp fragment, which has the sense orientation of integrase, includes the sequence of bases 1 to 1238 of SEQ ID NO: 27.

PCR analysis detected a 1238-bp fragment having the antisense version of integrase gene and the spacer sequence (FIG. 2, lanes 10-16) when primers having the sequence provided in SEQ ID NOs: 21 and 31 were used. The detected 1238-bp fragment, which has the antisense orientation of integrase, includes the sequence of bases 1233 to 2470 of SEQ ID NO: 27. Thus, PCR results showed the presence of the integrase dsRNA expression cassette in the transgenic plants transformed with a M. incognita integrase dsRNA expression cassette.

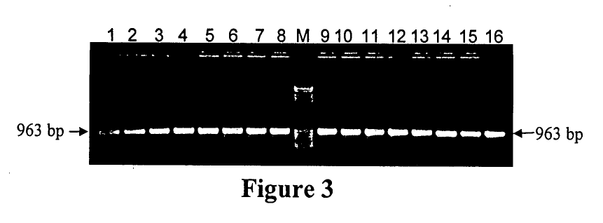

DNA from transgenic plants transformed with a M. incognita splicing factor dsRNA expression cassette was analyzed by PCR. Amplification using primers having the sequence provided in SEQ ID NOs: 15 and 32 detected a 963-bp fragment having the sense version of the splicing factor gene and the spacer sequence (FIG. 3, lanes 1-8). The detected 963-bp fragment having the sense orientation of the splicing factor gene includes bases 1 to 963 of SEQ ID NO: 28.

PCR detected a 963-bp fragment having the antisense version of the splicing factor gene and the spacer sequence (FIG. 3, lanes 10-16) when PCR was performed using primers having the sequence provided in SEQ ID NOs: 17 and 31. The 963-bp fragment carrying the antisense orientation of the splicing factor gene includes bases 356 to 1318 of SEQ ID NO: 28. Thus, PCR results showed the presence of the splicing factor dsRNA expression cassette in the transgenic plants transformed with a M. incognita splicing factor dsRNA expression cassette.

### Example 13

Bioassays Confirming that Transgenic Plants have Increased Resistance to Nematodes

To determine whether transgenic plants described in Example 12, which had nucleotide sequences for integrase or for the splicing factor of M. incognita, could resist nematode infection, the transgenic plants were inoculated with M. incognita juveniles.

All plant infection experiments were performed under standard green house conditions. M. incognita from a local field was maintained on tomato plants and eggs were hatched on moist filter paper. Approximately 2,500 freshly hatched juveniles were transferred to the soil close to the roots. The roots were washed off the soil and examined for infection 45 days later. The nematodes were dissected out of the roots with the help of forceps and a stereomicroscope.

While all the control plants (n=12) developed several large root knots, only a few of the transgenic plants (2 out of 25) expressing the splicing factor dsRNA formed root knots. The knots formed in the transgenic plants were significantly smaller in size as well as number when compared with knots formed in the control plants (FIGS. 1A-D and Table 1). Rest of the transgenic plants tested (23 out of 25) did not produce any visible knots.

Similar results were obtained with the transgenic plants expressing integrase dsRNA: 6 out of 19 transgenic plants produced root knots. Similarly, number of knots in each transgenic plant was lesser than in the control plants. Further the knots that formed in the transgenic plants were smaller. Moreover, 13 out of 19 transgenic did not form any root knots. Thus, our results show that the transgenic plants having dsRNA expression cassette for splicing factor or integrase gene of M. incognita were protected against infection by this parasite/pathogen.

### Feeding on Transgenic Plants Impairs Nematode Development

Further, the few root knots that were formed on the transgenic plants were tested. For this, the root knots were dissected and examined using a dissecting stereomicroscope. In control plants, several female nematodes were found in all the root knots. The worms in the control plants demonstrated characteristics of M. incognita, i.e., these nematodes were saccate (tear drop-shaped) and dark (FIG. 1C). In contrast, only a small fraction of the root knots of the transgenic plants had worms. Even in the few root knots in the transgenic plants, only one or two worms were found (Table 1). The worms from the transgenic plants were fusiform (elongated) and transparent (FIG. 1D). Since the dark color of healthy females in control plants is due to the dark granules of intestinal cells, the transparent body of nematodes in the transgenic knots probably indicates a lack of gut granules.

As seen in Table 1, the number of egg masses in the transgenic plants was lower as compared to the untreated control plants (See, Table 1, column 4). Further, the number of eggs hatching from each egg mass was greatly reduced in the transgenic plants as compared to the control plants (Table 1, column 5). These results indicate that the expression of dsRNA in host plants interferes with the normal development of the nematode.

TABLE 1
Effect of dsRNA expression in the host plant
on M. incognita infection.

| | | | Number of | Number of |
|---|---|---|---|---|
| | Number of | Number of | egg | eggs hatching/ |
| dsRNA | knots/plant | females/knot | masses/plant | egg mass |
| No dsRNA | 130 (12) | 9 (10) | 840 (5) | 97 (70) |
| Splicing | 3.5^{a} (25) | 4^{a} (50) | 2.5 (25) | 0.25 (20) |
| factor | | | | |
| Integrase | 12.6^{b} (19) | 6^{b} (50) | 9.8 (19) | 0.75 (20) |

The number of plants, knots or egg masses examined in each case are shown in parentheses.
^{a}knot size was considerably smaller than the control (see FIG. 1 A and 1B).
^{b}Color and shape of these worms were very different from the control (see FIG. 1C and 1D).

### Double-Stranded RNA Produced in Host Plants Induced RNAi in the Parasite

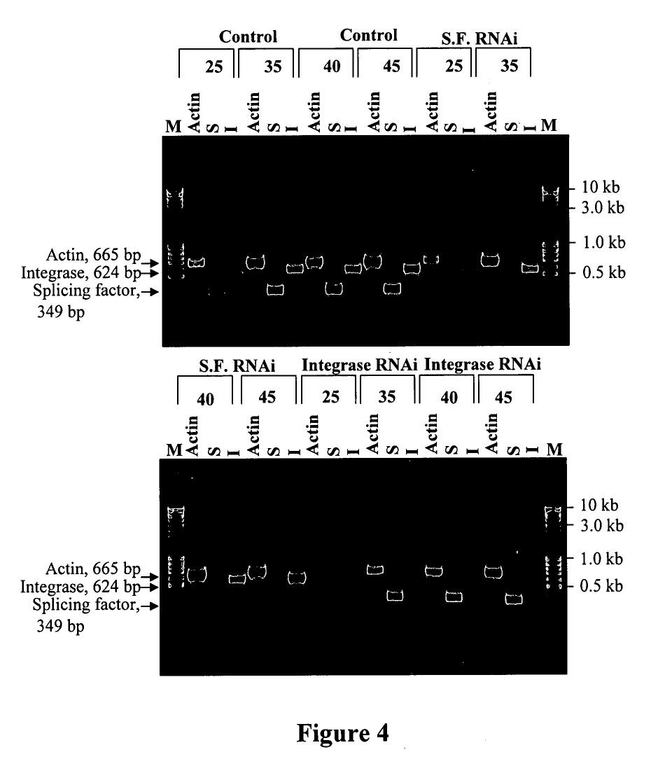

To determine whether the inability of nematodes to infect transgenic roots was a result of RNAi of the targeted genes, the presence of target mRNAs in the worms of transgenic root knots was analyzed by RT-PCR (FIG. 4).

Even 45 cycles of amplification with primers having the sequence provided in SEQ ID NOs: 15 and 16 failed to amplify the splicing factor cDNA from the RNA isolated from worms of transgenic plants that express splicing factor dsRNA (FIG. 4, see lanes S under the label S.F.RNAi). However, cDNAs of actin and integrase could be amplified from the same RNA preparations (FIG. 4, lanes “Actin” and “I” under the label S.F.RNAi). Primers having the sequence provided in SEQ ID NOs: 19 and 20 were used for detection of integrase DNA. Primers having the sequence provided in SEQ ID NOs: 36 and 37 were used to detect actin. Sequence of the 665-bp amplified actin product is provided in SEQ ID NO: 38.

Similarly, the actin and splicing factor cDNAs, but not the integrase cDNA, could be amplified from the worms of integrase transgenic plants. In contrast, all three cDNAs could be amplified from the worms of control plants (FIG. 4, lanes labeled “Control”). From these results, it is concluded that the failure to amplify cDNAs of target mRNAs was due to the absence of these mRNAs in the respective worms and not due to their recalcitrance for RT-PCR amplification or RNA degradation. Specific absence of the target mRNAs, which is a hallmark of RNAi response (Fire A, Xu S, Montgomery M K, Kostas S A, Driver S E, Mello C C, Potent and specific genetic interference by double-stranded RNA in Caenorhabditis elegans, Nature 1998; 391:806-811), therefore, the results indicate that the host plant-generated dsRNA had triggered RNAi in the parasitic nematode.

What is claimed is:

1. A transgenic plant comprising a nucleic acid molecule of a pathogen, whereby the transgenic plant has increased resistance to infection by the pathogen,
wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) a combination of (a) and (b) further comprising a spacer nucleotide sequence; and,
wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

2. The transgenic plant of claim 1, wherein the polynucleotide is DNA.

3. The transgenic plant of claim 1, wherein the polynucleotide is RNA.

4. The transgenic plant of claim 1, wherein (c) is double stranded RNA.

5. The transgenic plant of claim 1, wherein (d) is double stranded RNA.

6. A seed produced by the transgenic plant of claim 1.

7. A progeny of the transgenic plant of claim 1.

8. The transgenic plant of claim 1, wherein the protein involved in chromatin remodeling in the pathogen is an integrase.

9. The transgenic plant of claim 1, wherein the protein in the recombination pathway of the pathogen is an integrase.

10. The transgenic plant of claim 1, wherein the protein in the nucleotide repair pathway of the pathogen is an integrase.

11. The transgenic plant of claim 1, wherein the protein for post-transcriptional processing of RNA in the pathogen is a splicing factor.

12. The transgenic plant of claim 1, wherein the nucleic acid molecule is substantially homologous to a member selected from the group consisting of SEQ ID NO: 1, SEQ ID NO: 27, SEQ ID NO: 2, SEQ ID NO: 28, SEQ ID NO: 29, SEQ ID NO: 30 and combinations thereof.

13. The transgenic plant of claim 1, wherein the protein of the pathogen is a portion of an integrase protein comprising SEQ ID NO: 3.

14. The transgenic plant of claim 1, wherein the protein of the pathogen is a portion of a splicing factor protein comprising SEQ ID NO: 4.

15. The transgenic plant of claim 1, wherein the transgenic plant is a monocotyledonous plant.

16. The transgenic plant of claim 1, wherein the transgenic plant is a dicotyledonous plant.

17. The transgenic plant of claim 1, wherein the transgenic plant is selected from the group consisting of tomato, egg plant (brinjal), chilli (green pepper), avocado, cotton, corn, pulses, peanut (ground nut), soybean, pigeon pea, chick pea, field pea, rice, wheat, oat, barley, rye, sorghum, millet, coffee, cocoa, tea, citrus, date palm, banana, pineapple, pomegranate, persimmon, papaya, mango, guava, kiwi, cotton, tobacco, sugarcane, potato, carrot, yam, taro, sweet potato, cassava, black pepper, turmeric, cardamom, and combinations thereof.

18. The transgenic plant of claim 1, wherein the pathogen is a nematode.

19. The transgenic plant of claim 1, wherein the transgenic plant is a host for a pathogen belonging to the family selected from the group consisting of Tylenchoidea, Longidoridae and Trichodorideae.

20. The transgenic plant of claim 1, wherein the transgenic plant is a host for a pathogen of the genera selected from the group consisting of Meloidogyne, Globodera, Heterodera, Pratylenchus and Radopholus.

21. The transgenic plant of claim 1, wherein the transgenic plant is a host for a species of the pathogen of the genus Meloidogyne selected from the group consisting of M. incognita, M. hapta, M. javanica, M. chitwoodi, M. arenaria, M. nassi, M. graminicola, M. fallax and M. artiella.

22. The transgenic plant of claim 21, wherein the pathogen is M. incognita.

23. The transgenic plant of claim 21, wherein the transgenic plant is resistant to infection caused directly or indirectly by M. incognita.

24. A transgenic plant comprising a nucleic acid molecule of a nematode, whereby the transgenic plant has increased resistance to infection by the nematode,
wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) a combination of (a) and (b) further comprising a spacer nucleotide sequence; and,
wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

25. The transgenic plant of claim 24,
wherein the protein of the pathogen is selected from a group consisting of an integrase, a splicing factor, and combinations thereof.

26. A method of producing a transgenic plant, wherein the transgenic plant has increased resistance to infection by a pathogen,
wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is a combination of (a) and (b) further comprising a spacer nucleotide sequence, and
wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

27. The method of producing a transgenic plant of claim 26, wherein the method comprises:
transforming a plurality of first plants with a nucleic acid molecule of the pathogen to produce a plurality of second plants; and
selecting the transgenic plant from the plurality of second plants.

28. The method of claim 26, wherein the method further comprises transforming a first plant with an intermediary host comprising the nucleic acid of the pathogen,
wherein the intermediary host is selected from the group consisting of Agrobacterium tumefaciens and E. coli.

29. The method of claim 27 further comprising transforming a first plant with an Agrobacterium tumefaciens, wherein the method comprises:
obtaining an explant from the first plant,
constructing a recombinant vector comprising the nucleic acid of the pathogen,
mobilizing the recombinant vector in an Agrobacterium tumefaciens cell to produce a recombinant Agrobacterium tumefaciens cell,
co-cultivating the explant of the first plant with the recombinant Agrobacterium cell to produce a second plant, and
culturing the second plant to produce the transgenic plant.

30. A method of producing a transgenic plant comprising a nucleic acid molecule of a pathogen, whereby the transgenic plant has increased resistance to infection by the pathogen, wherein the method comprises:
injuring a plurality of leaves of a first plant to form a plurality of injured leaf discs;
placing the plurality of injured leaf discs on an agar-medium containing antibiotic;
adding an Agrobacterium onto the plurality of injured leaf discs to produce a plurality of transformed leaves, wherein the Agrobacterium comprises the nucleic acid molecule of the pathogen;
growing the plurality of transformed leaves to produce a plurality of second plants;
testing the plurality of second plants for the presence of the nucleic acid molecule of the pathogen to obtain a plurality of tested second plants;
selecting a transgenic plant from the plurality of tested second plants, wherein the transgenic plant comprises the nucleic acid sequence of the pathogen;
infecting the transgenic plant with the pathogen; and
confirming that the transgenic plant has increased resistance to infection by the pathogen.

31. A recombinant vector comprising a nucleic acid molecule of a pathogen,
wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is a combination of (a) and (b) further comprising a spacer nucleotide sequence, and
wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

32. The recombinant vector of claim 31, further comprising the nucleic acid molecule of the pathogen operably linked to a promoter, and
wherein the promoter is selected from a group consisting of a constitutive promoter, a tissue specific promoter, an inducible promoter, a wound inducible promoter, and combinations thereof.

33. The recombinant vector of claim 31, further comprising at least one selectable marker.

34. A transgenic plant comprising the recombinant vector of claim 31.

35. The recombinant vector of claim 31, wherein the recombinant vector is selected from the group consisting of pBC16, pBC20, pBC17 and pBC21.

36. An intermediary host cell comprising a recombinant vector,
wherein the recombinant vector comprises a nucleic acid molecule of a pathogen,
wherein the nucleic acid molecule of the pathogen is substantially homologous to a member selected from the group consisting of (a) a portion of a polynucleotide coding for a protein of the pathogen; (b) a complementary sequence to (a); (c) a combination of (a) and (b); and (d) is (c) further comprising a spacer nucleotide sequence, and
wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

37. The intermediary host of claim 36, selected from the group consisting of Agrobacterium tumefaciens and E. coli.

38. An isolated nucleic acid sequence comprising a nucleic acid molecule of a pathogen, wherein the nucleic acid of the pathogen is substantially homologous to a combination of (a) and (b), wherein (a) is a portion of a polynucleotide coding for a protein of the pathogen, and (b) is a sequence complementary to (a), and
wherein the protein of the pathogen is selected from a group consisting of a protein involved in chromatin remodeling in the pathogen, a protein in a recombination pathway of the pathogen, a protein in a nucleotide repair pathway of the pathogen, a protein for post transcriptional processing of RNA in the pathogen, and combinations thereof.

39. The isolated nucleic acid sequence of claim 38 further comprising a spacer nucleotide sequence.

40. The isolated nucleic acid sequence of claim 38, wherein the protein of the pathogen is selected from the group consisting of an integrase, a splicing factor, and combinations thereof.

41. The isolated nucleic acid sequence of claim 38, wherein the polynucleotide has at least 95% identity to a member selected from the group consisting of SEQ ID NO: 1, SEQ ID NO: 2, SEQ ID NO: 27, SEQ ID NO: 28, SEQ ID NO: 29, SEQ ID NO: 30, and combinations thereof.

42. The isolated nucleic acid sequence of claim 38, wherein the protein of the pathogen has at least 95% identity to a member selected from the group consisting of SEQ ID NO: 3, SEQ ID NO: 4, and combinations thereof.


SEQUENCE LISTING (38 sequences)

SEQ ID NO: 1 - DNA, 624 bp, Meloidogyne incognita
atgtcaaaggcaacgtatggaccaaaacccgagtcttttgtgttggacgatggacagcggtattatgttggagctgatgttggaatgtaccttaaatatcatcgtggaaccctttacaagcgctatcctcaactatggaaaagaatggcttctgtagatgaaaagaagaaaattcaagaattgggatgcgcaacttcctatatcaattctaatattatgctggttaaagctagtgaagttgaagaaatatttgaaggccaagaagagaaatatcgctctggaggtgttggctctggaaattatagccgttcagatgccgcaaatattaaaaatcgtggaggtccagccccttggcttacccagcaggtcaccagtggctctcatcatctcgaatctgttccgtgttctactccatcggcccatagacgtggacattttcgaaatcgtgattatgcttatgctgccgatgatttggaaaattataaacgtgttttgcaaaatgcagagactgctgaatccttggttccgattaggcttgatatggagttggacaatattaaacttcgtgattatttttgctacaacaagaacgacaaattaatgacccctgaaatgattgctgaa

SEQ ID NO: 2 - DNA, 349 bp, Meloidogyne incognita
ctgctcttttcgttgcacgtaatggtgttgaatttgaaaataaaattcgggaaagagaagcttcaaatatacgttttaattttcttacacctgtcgacccgtaccatgcttattacaaaaataaagtgaaggaattcgagactggagttgcttctgctgaattattaactaaacttaaaattcctgatgctgttcgtgaacatatacaaaaggctgaatttgttccaaaaaatccgccaaaaccttttgaattttgtgctgatccatcgacgataaacgcttttgatttagatcttatccaacttaccgctctatttgttgcaagaaatggacgtcaatttctcacaca

SEQ ID NO: 3 - protein, 208 aa, ARTIFICIAL
  SYNTEHTIC SEQUENCE
MSKATYGPKPESFVLDDGQRYYVGADVGMYLKYHRGTLYKRYPQLWKRMASVDEKKKIQELGCATSYINSNIMLVKASEVEEIFEGQEEKYRSGGVGSGNYSRSDAANIKNRGGPAPWLTQQVTSGSHHLESVPCSTPSAHRRGHFRNRDYAYAADDLENYKRVLQNAETAESLVPIRLDMELDNIKLRDYFCYNKNDKLMTPEMIAE

SEQ ID NO: 4 - protein, 115 aa, ARTIFICIAL
  SYNTHETIC SEQUENCE
ALFVARNGVEFENKIREREASNIRFNFLTPVDPYHAYYKNKVKEFETGVASAELLTKLKIPDAVREHIQKAEFVPKNPPKPFEFCADPSTINAFDLDLIQLTALFVARNGRQFLT

SEQ ID NO: 5 - DNA, 590 bp, Meloidogyne incognita
attttaattttattcaattctaaaatgtcaaaggcaacgtatggaccaaaacccgagtcttttgtgttggacgatgggcagcggtattatgttggtgctgatgttggaatgtacctcaaatatcatcgtggaaccctttacaagcgctatcctcagctttggaaaagaatggcctctgtagatgaaaagaagaaaattcaagaattgggatgcgcgacttcctatattaattctaatattatgctggtcaaagctagtgaagtagaagaaatctttgaaggccaagaagagaaatatcgctctggaggtgttggctctggaaattatagccgttcagatgccgcaaatattaaaaatcgtggaggtccaaccccttggcttacacaacaggtcaccagtggttctcatcatctcgaatctgtcccttgctccactccatcggcccatagacgtggacattttcgaaatcgcgattatgcttatgctgccgatgacttggaaaattataaacgtgtgttgcaaaatgcagagactgctgaatccttggttccgattaggctcgatatggagttggacaatattaaacttcg

SEQ ID NO: 6 - DNA, 629 bp, Heterodera glycines
tcaaaacgtacgggccaaggcccgagtcgttcacgcttgacgacggtgaacgttattatgtcggtgcggacgtgggccagtacttgaaataccatcgcggcacgctgtacaaacgctacccccaactgtggaagcgaatggcatcggtggaggagaagaagaagatccaggagttgggctgtgccacctcctacatcaactcgaacattatgctggtcaaagcgaacgaggttgaagacatttttgacggacaagaggagaaatatcgcgcgtcggtcagcggcggcagtgcgtacggacgcgcggagggtgcgcaaacgctgcgaagtcggggcagtgctccgtggctgaaccagcaggtgaccagcggttcccaccacctcgagtccgtgccctgctccaccccttcggcgcacgggcgaggccatttcaaaaatcgcgactacgcttacgtggcggatgacctcgagtcgtatcgccgggtgatggaaaatgcgcaggccggcgaatgtttggtgcccattcggctggacatggaattggacaatattaaactgcgggactgcttttgctacaacaaaaatgacaaactgatcacacccgaaatggttgcagaagtgttgtgtgaa

SEQ ID NO: 7 - DNA, 474 bp, Meloidogyne paranaensis
ggtttaattacccatttttgagattttaattttatttaatcctaaaatgtcaaaagcaacgtatggaccaaaacccgagtcttttgtgttggacgatggacagcggtattatgttggagctgatgttggaatgtaccttaaatatcatcgtggaaccctttacaagcgctatcctcaactatggaaaagaatggcttctgtagatgaaaagaagaaaattcaagaattgggatgcgcaacttcctatatcaattctaatattatgctggttaaagctagtgaagttgaagaaatatttgaaagccaaaaaaagtcccctataagtcaatagtaccctctggtagatcaatttgaatgggatatgagcgatccaaaaaatagtcctgaagaatttggacaaaaattgtctgcagaattgggtttgggcggagaatttgttgctgctattgcttattctattacaggtcaattatc

SEQ ID NO: 8 - DNA, 556 bp, Strongyloides ratti
atctctttaattaaaatacattcctaatataataataataataatgtctattgttccatatggagaaaaacctaagtcatttactttgtcagatggtgggacgtattttattggctctgaagttggagcttacctaaggttacatcgaggaacgttatataaaaaatttcctggattatggaaacaaatggctacatctgaacaaaggaaagaaattcaagaacttcaactttctccttcatatttatgttcaaatattatgttagtaaaagcagatgaagttgaagatttattaaacgggcaggaagaaaaatatagaacagcttctcaaagggatacaccttcaaagattgatgtatctataatttctaaatcagttgttcgtaatccaaatactccatggcattcttctcaggtaagtacaggacaacaacatttagaagctctaccatgtgctcatccaccagcattagctaaagggcatatgaaagttcgggaatatttatattcttttgacgacagtgaacattatcgaagagttcttgagaatgctgaa

SEQ ID NO: 9 - DNA, 120 bp, Brugia malayi
agtttgagattaaactcggatattctcactgccatcatcatgccgaaaatgaaattctatggtgatcgaccgcagtcattttctattgatgattcgggggagcgttactacatcggtgat

SEQ ID NO: 10 - DNA, 463 bp, Meloidogyne incognita
gttgacaaaactgctcttttcgttgcacgtaatggtgttgaatttgaaaataaaattcgggaaagagaagcttcaaatatacgttttaattttcttacacctgtcgacccgtaccatgcttattacaaaaataaagtgaaggaattcgagactggagttgcttctgctgaattattaactaaacttaaaattcctgatgctgttcgtgaacatatacaaaaggctgaatttgttccaaaaaatccgccaaaaccttttgaattttgtgctgatccatcgacgataaacgcttttgatttgtaagttttataataataaagaccttggagaacaaggtaagataacaagggggaggggaacgactcaatccctcatgaattaatttttttaaccttgtttaagagatcttatccaacttaccgctctatttgttgcaagaaatggacgtcaatttctcacacaa

SEQ ID NO: 11 - DNA, 640 bp, Heterodera glycines
cagatcatggcgcaagagccgcccttgcaaattgtgattcccagattggatgatgataaaccgttggtgcgtcgaccgacgcctcaggcacaaccggcggctccagcggtgccggctgaactacgcgtgtccaatcgggaggaggacgcgcagaacatcgaaccgtcaatgagtggcaaacaaatagtcgggttgatcattccaccgcccgatattcgaacaattgtggacaaaacggcgctttttgtggctcgcaacggattggaatttgagatgaaaatcaaagagcgcgaggcttccaacatgcgtttcaactttctcaaccctaccgacccatactttgcctactatcgcaacaaggtcaatgaatttgagacgggcgttgcttcggcggacactcagtccaaagtgaagttgccggaggctgtccgtgagcatgtgcagcgggccgaatttattccgcggctgccgcccaaaccgttcgaattctgtgccgatcctttgacgctaaacgcattcgatttggatctcatccatttgaccgctttgtttgtggcgcggaacggccgtcaatttctcactcagctgatgaaccgggaggtgcgaaacttccagtttgatttcttaaagcctcaacact

SEQ ID NO: 12 - DNA, 647 bp, Heterodera glycines
tggcgcaagagccgcccttacaaattgtgattcccagattggatgatgataaaccgttggtgcgtcgaccgacgcctcaggcacaaccggcggctccagcggtgccggctgaactgcgcgtgtccaatcgggaggaggacgcgcagaacatcgaaccgtcaatgagtggcaaacaaatagtcgggttgatcattccaccgcccgacattcgaacaattgtggacaaaacggcgcttttcgtggctcgcaacggattggaatttgagatgaaaatcaaagagcgcgaggcttccaacatgcgtttcaactttctcaaccctaccgacccatattttgcctactatcgcaacaaggtcaatgaatttgagacgggcgttgcatcggcggacactcagtccaaagtgaagttgccggaggctgtccgtgagcatgtgcagcgggccgaatttattccgcggctgccgcccaaaccgttcgaattctgtgccgatcctttgacgctaaacgcattcgatttggatctcatccatttgaccgctttgtttgtggcgcggaacggccgtcaatttctcactcagctgatgaaccgtgaggtgcgaaacttccagttttgattcctaaagcctcaacactccaccttccaatac

SEQ ID NO: 13 - DNA, 291 bp, Meloidogyne hapla
ggtttattacctacgtttgagaaaattattcattttaattaacccaactgagaaaaatgcctccagtagaaaatttggtgacatcttcacagcagcctattaaggtttctggacgtgaagaagattcgatgaataaagaaccttcaatgagcggacagacaattgttggattaattattccaccgccagatattcgaacaattgttgacaaaactgctctttttgttgcacgaaatggtgttgaatttgaaaaaaaattcgagaaagagaagcttcaaatatacgttttaa

SEQ ID NO: 14 - DNA, 266 bp, Ascaris suum
tggtttctaatcgcgaggaggactccatgaacaatgagccctcgctgagtggacgtcagatcatcggcattatttacccgccacccgatattcgcactattgtcgataagacggcagcattcgtcgcaaggaatggcatcgatttcgaaaataaaattcgcgagaaggaggcttcgaatgcaagattcagctttctgagtgccaccgatccgtataatgcttactaccaacataaagtggtcgagttccgcgatgggaaagctagt

SEQ ID NO: 15 - DNA, 29 bp, ARTIFICIAL
  SYNTHETIC SEQUENCE
tctggatccctgctcttttcgttgcacgt

SEQ ID NO: 16 - DNA, 29 bp, ARTIFICIAL
  SYNTHETIC SEQUENCE
tctctcgagtgtgtgagaaattgacgtcc

SEQ ID NO: 17 - DNA, 29 bp, ARTIFICIAL
  SYNTHETIC SEQUENCE
tctgagctcctgctcttttcgttgcacgt

SEQ ID NO: 18 - DNA, 29 bp, ARTIFICIAL
  SYNTHETIC SEQUENCE
tctggtacctgtgtgagaaattgacgtcc

SEQ ID NO: 19 - DNA, 30 bp, ARTIFICIAL
  SYNTHETIC SEQUENCE
tctggatccatgtcaaaggcaacgtatgga

SEQ ID NO: 20 - DNA, 29 bp, ARTIFICIAL
  SYNTHETIC SEQUENCE
tctctcgagttcagcaatcatttcagggg

SEQ ID NO: 21 - DNA, 30 bp, ARTIFICIAL
  SYNTHETIC SEQUENCE
tctgagctcatgtcaaaggcaacgtatgga

SEQ ID NO: 22 - DNA, 29 bp, ARTIFICIAL
  SYNTHETIC SEQUENCE
tctggtaccttcagcaatcatttcagggg

SEQ ID NO: 23 - DNA, 35 bp, ARTIFICIAL
  SYNTHETIC SEQUENCE
tctggatccctcgaggcttaacaataggtacttcc

SEQ ID NO: 24 - DNA, 35 bp, ARTIFICIAL
  SYNTHETIC SEQUENCE
tctgagctcggtacctctgcttgacccctggcaat

SEQ ID NO: 25 - DNA, 596 bp, ARABIDOPSIS THALIANA
gcttaacaataggtacttccaaatcttcaattttgattctaaagattggtccttttactctgtttctcaatttgagttttaggtattctttgattttgtattggtttcattctaaatattcatcctttactcaacttctagataagggatttaggtattctcaaatttccgatttgattcctttactcgtttctagattggggttttaggaattaccagttgggggttttgcaatttgcgtaatcaaagaattttatttgttgtattgcttggtattgaagtttgtctctgtttctctacctcgtcatgtaatgtgcttagatccattaagtaaatgcttgtggatatttatgtagatggttaagagtgatcgtgatcagagtccttctcttatttaactgcattgcctgtgagttgtggtcctgaaggttgttgttattattgaattctatgtatgtatagattatgtcattggtctcatgtggtttttatgggtaacgtctttactaataatagcactatgcttctggattttgatctatgtgatctgtaacatttctagttggtgtgtctttgattgccaggggtcaagcaga

SEQ ID NO: 26 - DNA, 705 bp, NICOTIANA TABACCUM
tcctacacaatgtgaatttgaattagtttggtcatacggtatatcatatgattataaataaaaaaaattagcaaaagaatataatttattaaatattttacaccataccaaacacaaccgcattatatataatcttaattatcattatcaccagcatcaacattataatgattcccctatgcgttggaacgtcattatagttattctaaacaagaaagaaatttgttcttgacatcagacatctagtattataactctagtggagcttaccttttcttttccttcttttttttcttcttaaaaaaattatcactttttaaatcttgtatattagttaagcttatctaaacaaagttttaaattcatttcttaaacgtccattacaatgtaatataacttagtcgtctcaattaaaccattaatgtgaaatataaatcaaaaaaagccaaagggcggtgggacggcgccaatcatttgtcctagtccactcaaataaggcccatggtcggcaaaaccaaacacaaaatgtgttatttttaattttttcctcttttattgttaaagttgcaaaatgtgttatttttggtaagaccctatggatatataaagacaggttatgtgaaacttggaaaaccatcaagttttaagcaaaaccctcttaagaacttaaattgagcttcttttggggcatttttctagtgagaa

SEQ ID NO: 27 - DNA, 1868 bp, ARTIFICIAL
  SYNTHETIC SEQUENCE
ggatccatgtcaaaggcaacgtatggaccaaaacccgagtcttttgtgttggacgatggacagcggtattatgttggagctgatgttggaatgtaccttaaatatcatcgtggaaccctttacaagcgctatcctcaactatggaaaagaatggcttctgtagatgaaaagaagaaaattcaagaattgggatgcgcaacttcctatatcaattctaatattatgctggttaaagctagtgaagttgaagaaatatttgaaggccaagaagagaaatatcgctctggaggtgttggctctggaaattatagccgttcagatgccgcaaatattaaaaatcgtggaggtccagccccttggcttacccagcaggtcaccagtggctctcatcatctcgaatctgttccgtgttctactccatcggcccatagacgtggacattttcgaaatcgtgattatgcttatgctgccgatgatttggaaaattataaacgtgttttgcaaaatgcagagactgctgaatccttggttccgattaggcttgatatggagttggacaatattaaacttcgtgattatttttgctacaacaagaacgacaaattaatgacccctgaaatgattgctgaactcgaggcttaacaataggtacttccaaatcttcaattttgattctaaagattggtccttttactctgtttctcaatttgagttttaggtattctttgattttgtattggtttcattctaaatattcatcctttactcaacttctagataagggatttaggtattctcaaatttccgatttgattcctttactcgtttctagattggggttttaggaattaccagttgggggttttgcaatttgcgtaatcaaagaattttatttgttgtattgcttggtattgaagtttgtctctgtttctctacctcgtcatgtaatgtgcttagatccattaagtaaatgcttgtggatatttatgtagatggttaagagtgatcgtgatcagagtccttctcttatttaactgcattgcctgtgagttgtggtcctgaaggttgttgttattattgaattctatgtatgtatagattatgtcattggtctcatgtggtttttatgggtaacgtctttactaataatagcactatgcttctggattttgatctatgtgatctgtaacatttctagttggtgtgtctttgattgccaggggtcaagcagaggtaccttcagcaatcatttcaggggtcattaatttgtcgttcttgttgtagcaaaaataatcacgaagtttaatattgtccaactccatatcaagcctaatcggaaccaaggattcagcagtctctgcattttgcaaaacacgtttataattttccaaatcatcggcagcataagcataatcacgatttcgaaaatgtccacgtctatgggccgatggagtagaacacggaacagattcgagatgatgagagccactggtgacctgctgggtaagccaaggggctggacctccacgatttttaatatttgcggcatctgaacggctataatttccagagccaacacctccagagcgatatttctcttcttggccttcaaatatttcttcaacttcactagctttaaccagcataatattagaattgatataggaagttgcgcatcccaattcttgaattttcttcttttcatctacagaagccattcttttccatagttgaggatagcgcttgtaaagggttccacgatgatatttaaggtacattccaacatcagctccaacataataccgctgtccatcgtccaacacaaaagactcgggttttggtccatacgttgcctttgacatgagctc

SEQ ID NO: 28 - DNA, 1318 bp, ARTIFICIAL
  SYNTHETIC SEQUENCE
ggatccctgctcttttcgttgcacgtaatggtgttgaatttgaaaataaaattcgggaaagagaagcttcaaatatacgttttaattttcttacacctgtcgacccgtaccatgcttattacaaaaataaagtgaaggaattcgagactggagttgcttctgctgaattattaactaaacttaaaattcctgatgctgttcgtgaacatatacaaaaggctgaatttgttccaaaaaatccgccaaaaccttttgaattttgtgctgatccatcgacgataaacgcttttgatttagatcttatccaacttaccgctctatttgttgcaagaaatggacgtcaatttctcacacactcgaggcttaacaataggtacttccaaatcttcaattttgattctaaagattggtccttttactctgtttctcaatttgagttttaggtattctttgattttgtattggtttcattctaaatattcatcctttactcaacttctagataagggatttaggtattctcaaatttccgatttgattcctttactcgtttctagattggggttttaggaattaccagttgggggttttgcaatttgcgtaatcaaagaattttatttgttgtattgcttggtattgaagtttgtctctgtttctctacctcgtcatgtaatgtgcttagatccattaagtaaatgcttgtggatatttatgtagatggttaagagtgatcgtgatcagagtccttctcttatttaactgcattgcctgtgagttgtggtcctgaaggttgttgttattattgaattctatgtatgtatagattatgtcattggtctcatgtggtttttatgggtaacgtctttactaataatagcactatgcttctggattttgatctatgtgatctgtaacatttctagttggtgtgtctttgattgccaggggtcaagcagaggtacctgtgtgagaaattgacgtccatttcttgcaacaaatagagcggtaagttggataagatctaaatcaaaagcgtttatcgtcgatggatcagcacaaaattcaaaaggttttggcggattttttggaacaaattcagccttttgtatatgttcacgaacagcatcaggaattttaagtttagttaataattcagcagaagcaactccagtctcgaattccttcactttatttttgtaataagcatggtacgggtcgacaggtgtaagaaaattaaaacgtatatttgaagcttctctttcccgaattttattttcaaattcaacaccattacgtgcaacgaaaagagcaggagctc

SEQ ID NO: 29 - DNA, 624 bp, ARTIFICIAL
  SYNTHETIC SEQUENCE
ttcagcaatcatttcaggggtcattaatttgtcgttcttgttgtagcaaaaataatcacgaagtttaatattgtccaactccatatcaagcctaatcggaaccaaggattcagcagtctctgcattttgcaaaacacgtttataattttccaaatcatcggcagcataagcataatcacgatttcgaaaatgtccacgtctatgggccgatggagtagaacacggaacagattcgagatgatgagagccactggtgacctgctgggtaagccaaggggctggacctccacgatttttaatatttgcggcatctgaacggctataatttccagagccaacacctccagagcgatatttctcttcttggccttcaaatatttcttcaacttcactagctttaaccagcataatattagaattgatataggaagttgcgcatcccaattcttgaattttcttcttttcatctacagaagccattcttttccatagttgaggatagcgcttgtaaagggttccacgatgatatttaaggtacattccaacatcagctccaacataataccgctgtccatcgtccaacacaaaagactcgggttttggtccatacgttgcctttgacat

SEQ ID NO: 30 - DNA, 349 bp, ARTIFICIAL
  SYNTHETIC SEQUENCE
tgtgtgagaaattgacgtccatttcttgcaacaaatagagcggtaagttggataagatctaaatcaaaagcgtttatcgtcgatggatcagcacaaaattcaaaaggttttggcggattttttggaacaaattcagccttttgtatatgttcacgaacagcatcaggaattttaagtttagttaataattcagcagaagcaactccagtctcgaattccttcactttatttttgtaataagcatggtacgggtcgacaggtgtaagaaaattaaaacgtatatttgaagcttctctttcccgaattttattttcaaattcaacaccattacgtgcaacgaaaagagcag

SEQ ID NO: 31 - DNA, 20 bp, ARTIFICIAL
  SYNTHETIC SEQUENCE
gcttaacaataggtacttcc

SEQ ID NO: 32 - DNA, 20 bp, ARTIFICIAL
  SYNTHETIC SEQUENCE
tctgcttgacccctggcaat

SEQ ID NO: 33 - DNA, 29 bp, ARTIFICIAL
  SYNTHETIC SEQUENCE
tcttctagatcctacacaatgtgaatttg

SEQ ID NO: 34 - DNA, 29 bp, ARTIFICIAL
  SYNTHETIC SEQUENCE
tcttctagattctcactagaaaaatgccc

SEQ ID NO: 35 - DNA, 835 bp, ARTIFICIAL
  SYNTHETIC SEQUENCE OF 35S PROMOTER
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacg

SEQ ID NO: 36 - DNA, 20 bp, ARTIFICIAL
  SYNTHETIC SEQUENCE
catcgcaatggtcgaagaag

SEQ ID NO: 37 - DNA, 20 bp, ARTIFICIAL
  SYNTHETIC SEQUENCE
caacatagcaaagcttctcc

SEQ ID NO: 38 - DNA, 665 bp, ARTIFICIAL
  SYNTHETIC SEQUENCE OF ACTIN PROMOTER
catcgcaatggtcgaagaagtagctgctcttgttgttgataatggctcaggcatgtgcaaggctggatttgcgggtgatgatgctcctcgtgccgtttttccaagtattgtcggtcgtcctcgacatcaaggagttatggttggaatgggtcaaaaggattcgtatgtgggagatgaagctcaaagcaaaagaggtatcttgactttgaaatacccgattgagcacggaattgtaacaaactgggacgacatggagaagatttggcatcatacgttctacaatgagttgcgtgttgctcctgaagaacatccagttctccttacagaggcacctctcaacccaaaggctaaccgtgaaaagatgactcaaatcatgtttgagacattcaacacgcctgccatgtatgttgccatccaagccgttctttctttgtatgcttctggacgtactactggaattgttcttgactctggagatggtgttacgcacacagttccaatttatgaaggttatgctttgccacacgcaatcttgcgtcttgacttggcaggacgtgatttgaccgactacttgatgaagatcctcactgaacgtggttattctttcaccacaaccgccgaacgtgagattgtccgtgacatcaaggagaagctttgctatgttg